SEQUENCE LISTING (19 sequences)

SEQ ID NO: 1 - DNA, 53 bp, Homo sapiens
taacatattgatgaaacagagcaaaaacgcctagaaatagatccaaataacag

SEQ ID NO: 2 - DNA, 53 bp, Homo sapiens
agagtgcagtggcgcgatctcggctcagctgcaagctctgcctgccgggttca

SEQ ID NO: 3 - DNA, 53 bp, Homo sapiens
tggcacttcagtctctgaaaccctgcagcactgaggggtcagtcagctgggca

SEQ ID NO: 4 - DNA, 53 bp, Homo sapiens
aaacacatttaccagataagtaaggcactaaagtaaattccccggtctgggaa

SEQ ID NO: 5 - DNA, 53 bp, Homo sapiens
ggtgctggctttgctggagctggcgcaggcaggaccacggtgctctggactgc

SEQ ID NO: 6 - DNA, 53 bp, Homo sapiens
tctgggcttggggcttcctaactgctagcaaatactcttaggaagagaaacca

SEQ ID NO: 7 - DNA, 53 bp, Homo sapiens
tgtgaaaaagttaaacattgaagtaactgaatttttatgatattccccccact

SEQ ID NO: 8 - DNA, 53 bp, Homo sapiens
tcacagactcaggagatggcgttggcagaaatcacttggtcccactgggattc

SEQ ID NO: 9 - DNA, 62 bp, Homo sapiens
agggcatggatttccagggccctggggagacgctgagccctcgctcctcatggtgtgcctcc

SEQ ID NO: 10 - DNA, 53 bp, Homo sapiens
gagataaggcaggaggccatttacagctagtcttggggaggctgaatcagagc

SEQ ID NO: 11 - DNA, 53 bp, Homo sapiens
atgatgctgattccacttcagttgaaagctatgtactctgttgctagtcaatg

SEQ ID NO: 12 - DNA, 53 bp, Homo sapiens
gtccaagattgaagggctgcatctggatgagggtcatctggctacattataac

SEQ ID NO: 13 - DNA, 53 bp, Homo sapiens
gttatttttacgctgtcttctgtgaaaggttttgagaatgaaatgagacagag

SEQ ID NO: 14 - DNA, 53 bp, Homo sapiens
aagtaaaataagggttactgaacacaaggcactgctatatggtaactgttgat

SEQ ID NO: 15 - DNA, 53 bp, Homo sapiens
caagtgaaggttcctgggccgaattcctttttcagtcactttaaggaacagga

SEQ ID NO: 16 - DNA, 53 bp, Homo sapiens
ttgtttcaatgttgtttaaggtaagactttgataactttggatttgtttgaag

SEQ ID NO: 17 - DNA, 53 bp, Homo sapiens
aagcagagtgtttccagggctgtttgacgcaatagagggctgacaccaatagg

SEQ ID NO: 18 - DNA, 53 bp, Homo sapiens
agctgcgcctggaaagcgagctcggggtgggtgcctacagcagggtgcgcccg

SEQ ID NO: 19 - DNA, 53 bp, Homo sapiens
atgtgatcatgtgtctcaccctttcaaggctgaaagcaacagtgcagacgatg

(19) United States
(12) Patent Application Publication
(10) Pub. No.: US 2013/0116139 A1
(43) Pub. Date: May 9, 2013

(54) INNATE IMMUNITY MARKERS OF CANCER

(71) Applicants: La Creis Renee Kidd, Louisville, KY (US); Kevin Sean Kimbro, Durham, NC (US)

(72) Inventors: La Creis Renee Kidd, Louisville, KY (US); Kevin Sean Kimbro, Durham, NC (US)

(21) Appl. No.: 13/582,170

(22) PCT Filed: Mar. 11, 2011
(86) PCT No.: PCT/US2011/028174

§ 371 Date: Dec. 13, 2012

Related U.S. Application Data
(60) Provisional application No. 61/313,595, filed on Mar. 12, 2010.

(51) Int. Cl. C12Q 1/68

(52) U.S. Cl. CPC C12Q 1/6886
USPC 506/9; 435/6.11

(57) ABSTRACT

This invention relates generally to methods of determing if a subject has a genetic predisposition to cancer, e.g., prostate cancer and breast cancer.

### CROSS REFERENCE TO RELATED APPLICATION

This application claims priority to U.S. Application No. 61/313,595, filed on Mar. 12, 2010, the entire contents of which are hereby incorporated by reference in its entirety.

### STATEMENT AS TO FEDERALLY SPONSORED RESEARCH

This invention was made with government support under Grant No. T32 ES011564 awarded by the National Institute of Environmental Health Sciences. The government has certain rights in the invention.

### TECHNICAL FIELD

The invention relates to genetic markers of cancer and methods of use thereof.

### BACKGROUND

Evolutionary conserved toll-like receptors are essential to the body's alarm system in response to bacterial and viral infections (Latz et al., J Biol Chem 2002, 277:47834-47843; Frantz et al., Nat Clin Pract Cardiovasc Med 2007, 4:444-454; Tsujimoto et al., Shock 2008, 29:315-321). TLRs (TLR 1-9) serve as pathogen sensors and transducers of pathogen-activated signaling pathways. This multi-tier cascade is involved in the production of inflammatory cytokines and chemokines essential to tumor growth. Pathogen recognition is regulated by a complex network of extracellular accessory protein (CD-14, MD-2) that interact with the pathogen and in turn binds to pattern recognition receptors (TLR 1-10) and transmit a signal through adaptor molecules (MyD88, TRAM, TRIF) and downstream targets (TANK, BTK, TOLLIP, IRAKs). This signaling cascade ultimately leads to the activation of Nuclear Factor-kappa Beta (NFkB) and Interferon Regulatory Factors (IRF 3, 5, 7) that induce cytokine, chemokine, and interferon production (Latz et al., J Biol Chem 2002, 277:47834-47843; Akira et al., Biochem Soc Trans 2003, 31(Pt 3):637-642; Beutler, Immunogenetics 2005, 57:385-392; Beutler, Annu Rev Pharmacol Toxicol 2003, 43:609-628; Gay and Keith, Nature 1991, 351:355-356; Gay et al., FEBS Lett 1991, 291:87-91; Nakata et al., Cell Microbiol 2006, 8:1899-1909; Muzio et al., J Leukoc Biol 2000, 67:450-456; Jin et al., Cell 2007, 130:1071-1082; Jin et al., Mol Vis 2007, 13:1953-1961; Farhat et al., J Leukoc Biol 2008, 83:692-701; Beutler et al., Adv Exp Med Biol 2005, 560:29-39). Recent efforts have demonstrated a link between cancer risk and innate immunity markers (Stevens et al., Int. J. Cancer, 2008. 123(11):2644-2650; Sun et al., J. Natl. Cancer Inst., 2005. 97(7):525-532; Cheng et al., Cancer Epidemiol Biomarkers Prey, 2007. 16(2):352-5), Mason et al., Prostate, 2009. 70(3):262-9), albeit equivocally (Cheng et al., Cancer Epidemiol Biomarkers Prev, 2007. 16(2):352-5).

### SUMMARY

The present invention is based, at least in part, on the discovery of the role of the innate immune system in cancer etiology and provides a prognostic and diagnostic panel of single nucleotide polymorphism (SNP) biomarkers for cancer, e.g., prostate cancer (PCa) and breast cancer (BCa).

In one aspect, the invention features methods of determining a subject's risk of developing cancer, e.g., PCa or BCa, the method comprising detecting the presence or identity of a haplotype in a sample from the subject, wherein the presence or identity of the haplotype indicates that the subject has an increased risk of developing cancer.

In one embodiment, the presence or identity of a haplotype comprising an “A” at rs4696480; a “G” at rs5743899; a “C” at rs4830807; a “T” at rs230528; or “C” at rs5743808 indicates that the subject has an increased risk of developing PCa.

In one embodiment, the presence or identity of a haplotype comprising an “A” at rs10025405; a “T” at rs4696480; a “T” at rs4251524; a “C” at rs7045953; a “C” at rs6442161; or a “C” at rs7251 indicates that the subject has an increased risk of developing BCa.

In one embodiment, detecting the presence or identity of a haplotype comprises obtaining a sample comprising DNA from the subject; and determining the identity, presence, or absence of the polymorphisms in the sample.

In one embodiment, the sample is obtained from the subject by a health care provider. In some embodiments, the sample is provided by the subject without the assistance of a health care provider.

In these methods, the subject can be of African descent (Africans, Carribeans, African-Americans); Asian descent (Chinese, Japanese, Indian, Pacific Islanders); or European Descent (non-hispanic Caucasians). As used herein, a subject of African descent is a subject having a West African Ancestry (WAA) score greater than 25%, e.g., greater than 30%, greater than 35%, greater than 40%, greater than 45%, greater than 50%, greater than 60%, greater than 70%, greater than 80, or greater than 90%. As used herein, a subject of European descent has a WAA score of less than 25%, e.g., less than 20%, less than 15%, less than 10%, or less than 5%.

TABLE A
SNPs Associated with Increased Prostate Cancer Risk

| | | Allele associated |
|---|---|---|
| | | with increased |
| SNP | Sequence | risk |
| rs4830807 | TAACATATTGATGAAACAGAGCAAAA[A/C] | C |
| | GCCTAGAAATAGATCCAAATAACAG | |
| | (SEQ ID NO: 1) | |
| rs37474 | AGAGTGCAGTGGCGCGATCTCGGCTC[A/G] | C |
| | CTGCAAGCTCTGCCTGCCGGGTTCA | |
| | (SEQ ID NO: 2) (complement) | |
| rs5743899 | TGGCACTTCAGTCTCTGAAACCCTGC[A/G] | G |
| | CACTGAGGGGTCAGTCAGCTGGGCA | |
| | (SEQ ID NO: 3) | |
| rs230528 | AAACACATTTACCAGATAAGTAAGGC[A/C] | T |
| | TAAAGTAAATTCCCCGGTCTGGGAA | |
| | (SEQ ID NO: 4) (complement) | |
| rs1052576 | GGTGCTGGCTTTGCTGGAGCTGGCGC[A/G] | G |
| | GCAGGACCACGGTGCTCTGGACTGC | |
| | (SEQ ID NO: 5) | |
| rs3024498 | TCTGGGCTTGGGGCTTCCTAACTGCT[A/G] | G |
| | CAAATACTCTTAGGAAGAGAAACCA | |
| | (SEQ ID NO: 6) | |
| rs1049216 | TGTGAAAAAGTTAAACATTGAAGTAA[C/T] | C |
| | GAATTTTTATGATATTCCCCCCACT | |
| | (SEQ ID NO: 7) | |

TABLE B
SNPs Associated with Increased
Breast Cancer Risk

| SNP | Sequence | |
|---|---|---|
| rs10025405 | TCACAGACTCAGGAGATGGCGTTGGC[A/G] | A |
| | AAATCACTTGGTCCCACTGGGATTC | |
| | (SEQ ID NO: 8) | |
| rs7251 | AGGGCATGGATTTCCAGGGCCCTGGGGAG | C |
| | A[C/G]CTGAGCCCTCGCTCCTCATGGTG | |
| | TGCCTCC(SEQ ID NO: 9) | |
| rs6442161 | GAGATAAGGCAGGAGGCCATTTACAG[C/T] | C |
| | AGTCTTGGGGAGGCTGAATCAGAGC | |
| | (SEQ ID NO: 10) | |
| rs4251545 | ATGATGCTGATTCCACTTCAGTTGAA[A/G] | T |
| | CTATGTACTCTGTTGCTAGTCAATG | |
| | (SEQ ID NO: 11) (complement) | |
| rs4696480 | GTCCAAGATTGAAGGGCTGCATCTGG[A/T] | T |
| | GAGGGTCATCTGGCTACATTATAAC | |
| | (SEQ ID NO: 12) | |
| rs7045953 | GTTATTTTTACGCTGTCTTCTGTGAA[A/G] | C |
| | GTTTTGAGAATGAAATGAGACAGAG | |
| | (SEQ ID NO: 13) (complement) | |

As used herein, an “allele” is one of a pair or series of genetic variants of a polymorphism at a specific genomic location. A “cancer susceptibility allele” is an allele that is associated with increased susceptibility of developing cancer.

As used herein, a “haplotype” is one or a set of signature genetic changes (polymorphisms) that are normally grouped closely together on the DNA strand, and are usually inherited as a group; the polymorphisms are also referred to herein as “markers.” A “haplotype” as used herein is information regarding the presence or absence of one or more genetic markers in a given chromosomal region in a subject. A haplotype can consist of a variety of genetic markers, including indels (insertions or deletions of the DNA at particular locations on the chromosome); single nucleotide polymorphisms (SNPs) in which a particular nucleotide is changed; microsatellites; and minisatellites.

“Linkage disequilibrium” refers to when the observed frequencies of haplotypes in a population does not agree with haplotype frequencies predicted by multiplying together the frequency of individual genetic markers in each haplotype.

The term “chromosome” as used herein refers to a gene carrier of a cell that is derived from chromatin and comprises DNA and protein components (e.g., histones). The conventional internationally recognized individual human genome chromosome numbering identification system is employed herein. The size of an individual chromosome can vary from one type to another with a given multi-chromosomal genome and from one genome to another. In the case of the human genome, the entire DNA mass of a given chromosome is usually greater than about 100,000,000 base pairs. For example, the size of the entire human genome is about 3×10⁹ base pairs.

The term “gene” refers to a DNA sequence in a chromosome that codes for a product (either RNA or its translation product, a polypeptide). A gene contains a coding region and includes regions preceding and following the coding region (termed respectively “leader” and “trailer”). The coding region is comprised of a plurality of coding segments (“exons”) and intervening sequences (“introns”) between individual coding segments.

The term “probe” refers to an oligonucleotide. A probe can be single stranded at the time of hybridization to a target. As used herein, probes include primers, i.e., oligonucleotides that can be used to prime a reaction, e.g., a PCR reaction.

Unless otherwise defined, all technical terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWING

FIG. 1 is a series of four line graphs showing the estimated statistical power under different genetic models.

FIG. 2 is a table of sequences of innate immunity SNPs.

FIG. 3 is a table of innate immunity SNPs and PCA risk models among MAD.

FIG. 4 is a table of innate immunity SNPs and PCA MDR modeling for MAD.

FIG. 5 is a table of innate immunity SNPs and aggressive disease among MAD.

FIG. 6 is a table of innate immunity SNPs associated with PCA risk among men of European descent.

FIG. 7 is a table of innate immunity and PCA among EA using MDR modeling.

FIG. 8 is a table of innate immunity SNPs and aggressive tumor grade among men of European descent.

FIG. 9 is a table of innate immunity SNPs and aggressive disease among Europeans using MDR Modeling.

### DETAILED DESCRIPTION

The methods described herein are based, at least in part, on the discovery of haplotypes and markers that are associated with increased risk of having or developing PCa or BCa. As described herein, analysis provided evidence of an association of the disclosed SNPs and haplotypes with these diseases.

### Prostate Cancer

Prostate cancer (PCa) accounts for 25% of all diagnosed cancer cases and is the second leading cause of cancer-related deaths in 2010 among all U.S. men (American Cancer Society, Cancer Facts & Figures 2010. 2010, Amerian Cancer Society: Atlanta). Men of African descent carry a substantial portion of the burden of this disease (American Cancer Society, Cancer Facts & Figures 2011-2012. 2011, Amerian Cancer Society: Atlanta). For instance, African-American men are 1.6- and 2.4-fold more likely to receive a prostate cancer diagnosis and die from the disease relative to their Caucasian counterparts, respectively (American Cancer Society, Cancer Facts & Figures for African Americans 2011-2012. 2011. pp. 10 and 12). African-American men are also more likely to be diagnosed at an advanced stage. Prostate cancer occurs less often in Asian-American and Hispanic/Latino men than in non-Hispanic whites. Prostate cancer is most common in North America, northwestern Europe, Australia, and on Caribbean islands. It is less common in Asia, Central America, and South America. The reasons for this are not clear. Lifestyle differences (e.g., diet) may be important: men of Asian descent living in the U.S. have a lower risk of prostate cancer than white Americans, but their risk is higher than that of men of similar backgrounds living in Asia.

### Breast Cancer

Breast cancer (BCa) is the second most common cancer in women, and is found in one in eight women in the United States. African-American women are more likely than all other women to die from breast cancer. Twice as many African-American women who have breast cancer die from the disease when compared to white women, although fewer African-American women get the disease. At least part of this seems to be because African-American women tend to have more aggressive tumors, although why this is the case is not known. Asian, Hispanic, and Native-American women have a lower risk of developing and dying from breast cancer. Asian women have some of the lowest breast cancer rates of any group in the world; however, the rates of breast cancer among Asians are approaching those of white women. Unlike the West where women typically present after age 50 with early stage disease, breast cancer in Asian women occurs at a younger age and is usually presented and diagnosed at a later stage of development. More patients present with locally-advanced Stage III disease in Asian countries than in the West. Further, while breast cancer mortality is declining in Europe and the U.S., in some areas, notably China, it is rising.

### Methods of Evaluating Susceptibility to PCa or BCa

Described herein are a variety of methods for determining susceptibility to PCa or BCa. “Susceptibility” does not necessarily mean that the subject will develop PCa or BCa, but rather that the subject is, in a statistical sense, more likely to develop PCa or BCa than an average member of the population, i.e., has an increased risk of developing PCa or BCa. As used herein, susceptibility to PCa or BCa exists if the subject has an allele or a haplotype associated with an increased risk of PCa or BCa as described herein. Ascertaining whether the subject has such an allele or a haplotype is included in the concept of determining susceptibility to PCa or BCa as used herein. Such determination is useful, for example, for purposes of diagnosis, treatment selection, and genetic counseling. Thus, the methods described herein can include detecting an allele or a haplotype associated with an increased risk of PCa or BCa as described herein for the subject.

As used herein, “detecting an allele or a haplotype” includes obtaining information regarding the identity, presence or absence of one or more genetic markers in a subject. Detecting an allele or a haplotype can, but need not, include obtaining a sample comprising DNA from a subject, and/or assessing the identity, presence or absence of one or more genetic markers in the sample. The individual or organization who detects the allele or haplotype need not actually carry out the physical analysis of a sample from a subject; the information can be obtained by analysis of the sample by a third party. Thus the methods can include steps that occur at more than one site. For example, a sample can be obtained from a subject at a first site, such as at a health care provider, or at the subject's home in the case of a self-testing kit. The sample can be analyzed at the same or a second site, e.g., at a laboratory or other testing facility.

Detecting an allele or a haplotype can also include or consist of reviewing a subject's medical history, where the medical history includes information regarding the identity, presence or absence of one or more genetic markers in the subject, e.g., results of a genetic test.

In some embodiments, to detect the presence of an allele or a haplotype described herein, a biological sample that includes nucleated cells (such as tissue, biopsy, formalin-fixed paraffin-embedded tissue, blood, a cheek swab, or mouthwash) is prepared and analyzed for the presence or absence of preselected markers. Such diagnoses may be performed by diagnostic laboratories, or, alternatively, diagnostic kits can be manufactured and sold to health care providers or to private individuals for self-diagnosis. Diagnostic or prognostic tests can be performed as described herein or using well known techniques, such as described in U.S. Pat. No. 5,800,998.

Results of these tests, and optionally interpretive information, can be returned to the subject, the health care provider or to a third party payor. The results can be used in a number of ways. The information can be, e.g., communicated to the tested subject, e.g., with a prognosis and optionally interpretive materials that help the subject understand the test results and prognosis. The information can be used, e.g., by a health care provider, to determine whether to administer a specific drug, or whether a subject should be assigned to a specific category, e.g., a category associated with a specific disease phenotype, or with drug response or non-response. The information can be used, e.g., by a third party payor such as a healthcare payer (e.g., insurance company or HMO) or other agency, to determine whether or not to reimburse a health care provider for services to the subject, or whether to approve the provision of services to the subject. For example, the healthcare payer may decide to reimburse a health care provider for treatments for PCa or BCa if the subject has an increased risk of developing PCa or BCa. As another example, a drug or treatment may be indicated for individuals with a certain haplotype, and the insurance company would only reimburse the health care provider (or the insured individual) for prescription or purchase of the drug if the insured individual has that haplotype. The presence or absence of the haplotype in a patient may be ascertained by using any of the methods described herein.

Information gleaned from the methods described herein can also be used to select or stratify subjects for a clinical trial. For example, the presence of a selected haplotype described herein can be used to select a subject for a trial. The information can optionally be correlated with clinical information about the subject, e.g., diagnostic or prognostic information.

### Innate Immunity Signaling, Sequence Variants, and Cancer Risk

Most studies that seek to explain disparities associated with complex diseases such as prostate and breast cancer place an emphasis on socio-economic, cultural, or environmental differences, while overlooking genetic-based etiological factors (Mason et al., Prostate, 2009. 70(3):262-9). However, emerging genome-wide association studies and replicated findings on the relationship between single nucleotide polymorphisms (SNPs) suggest genome variation plays an important, yet largely uncharacterized role in dissecting the genetic underpinnings of prostate and breast cancer health disparities (Salinas et al., Cancer Epidemiol. Biomarkers Prev., 2008. 17(5):1203-1213; Xu et al., Cancer Epidemiol. Biomarkers Prev., 2009. 18(7):2145-2149; Amundadottir et al., Nat. Genet., 2006. 38(6):652-658; Robbins et al., Genome Res., 2007. 17(12):1717-17224-7). Moreover, recent advances in molecular and genetic studies demonstrate a relationship between chronic/recurrent inflammation and complex diseases, including asthma and several cancers (Lazarus et al., Immunol. Rev., 2002. 190:9-25; De Marzo et al., Am. J. Pathol., 1999. 155(6):1985-1992; Lee et al., Clin. Cancer Res., 2005. 11(18):6431-6441). Regarding prostate and breast cancer, it is speculated that chronic or recurrent inflammation, attributed to persistent exposure to environmental factors (e.g., pathogens, such as microplasms or dietary carcinogens) or hormonal imbalances may alter the tissue microenvironment that favors tumor growth (Maitland and Collins, J Cell Biochem, 2008. 105(4):931-9; Vasto et al., Future Oncol, 2008. 4(5):637-45; Urisman et al., PLoS Pathog, 2006. 2(3):e25; Nakai et al., Cancer Lett, 2007. 251(1):164-7; De Marzo et al., Nat Rev Cancer, 2007. 7(4):256-69). The combination of bacterial or microbial (either pathogenic or commensals) may result in sustained injury of the prostate or breast and to the development of chronic inflammation and regenerative ‘risk factor’ lesions, referred to as proliferative inflammatory atrophy (De Marzo et al., Nat Rev Cancer, 2007. 7(4):256-69; El-Omar et al., Oncogene, 2008. 27(2):244-252). Consequently, genetic susceptibilities in innate immunity genes have been implicated in prostate and breast cancer. However, it remains to be determined whether genetic variations detected in subjects of African or Asian descent may contribute to higher PCa and BCa susceptibility and disease severity relative to their Caucasian counterparts.

As a hallmark of innate immunity, inflammation is the first line of defense in response to pathogens. Evolutionary conserved toll-like receptors are essential to the body's alarm system in response to bacterial and viral infections (Latz et al., J Biol Chem, 2002. 277(49):47834-43; Frantz et al., Nat Clin Pract Cardiovasc Med, 2007. 4(8):444-54; Tsujimoto et al., Shock, 2008. 29(3):315-21). TLRs (TLR 1-9) serve as pathogen sensors and transducers of pathogen-activated signaling pathways. This multi-tier cascade is involved in the production of inflammatory cytokines and chemokines essential to tumor growth. Following pathogen recognition, TLRs coupled with extracellular accessory proteins (CD-14, MD-2) transmit a signal through adaptor molecules (MyD88, TRAM, TRIF) and downstream targets Following pathogen recognition, TLRs coupled with extracellular accessory proteins (CD-14, MD-2) transmit a signal through adaptor molecules (MyD88, TRAM, TRIF) and downstream targets (TANK, BTK, TOLLIP, IRAKs). [TANK-binding Kinase 1 Bruton, agammaglobuinemia tyrosine kinase (BTK), toll-interacting protein (TOLLIP), Interleukin-1 associated kinases (IRAK1-4), tumor necrosis receptor-associated factor 6 (TRAF6), TRAF associated NFKB activator (TANK). This signaling cascade ultimately leads to the activation of Nuclear Factor-kappa Beta (NFkB) and Interferon Regulatory Factors (IRF 3, 5, 7), which in turn induce cytokine, chemokine and interferon production (Latz et al., J Biol Chem, 2002. 277(49):47834-43; Akira et al., Biochem Soc Trans, 2003. 31(Pt 3):637-42; Beutler, Immunogenetics, 2005. 57(6):385-92; Beutler, Annu Rev Pharmacol Toxicol, 2003. 43:609-28; Gay and Keith, Nature, 1991. 351(6325):355-6; Nakata et al., Cell Microbiol, 2006. 8(12):1899-909; Muzio et al., J Leukoc Biol, 2000. 67(4):450-6; Jin et al., Cell, 2007. 130(6):1071-82; Jin et al., Mol V is, 2007. 13:1953-61; Farhat et al., Leukoc Biol, 2008. 83(3):692-701; Beutler et al., Adv Exp Med Biol, 2005. 560:29-39).

Recent efforts have demonstrated a link between prostate cancer risk and innate immunity markers (Mason et al., Prostate, 2009. 70(3):262-9; Stevens et al., Int. J Cancer, 2008. 123(11):2644-2650; Sun et al., J. Natl. Cancer Inst., 2005. 97(7):525-532; Cheng et al., Cancer Epidemiol Biomarkers Prey, 2007. 16(2):352-5) albeit equivocally (Cheng et al., Cancer Epidemiol Biomarkers Prey, 2007. 16(2):352-5). Interestingly, a majority of these studies focused on the TLR1-6-10 cluster (Sun et al., J. Natl. Cancer Inst., 2005. 97(7):525-532; Zheng et al., Cancer Res., 2004. 64(8):2918-2922), TLR4 (Song et al., Cancer Genet Cytogenet, 2009. 190(2):88-92; Wang et al., Prostate, 2009. 69(8):874-85; Chen et al., Cancer Res., 2005. 65(24):11771-11778); IRAK1, 4 (Sun et al., Cancer Epidemiol. Biomarkers Prev., 2006. 15(3):480-485), or CD14 (Mason et al., Prostate, 2009. 70(3):262-9) sequence variants without consideration of other important downstream targets. For example, Zheng and co-workers (2004) revealed a 26% increase in PCa susceptibility among Swedish men who carried at least one TLR4_rs11381 C allele when compared to those with the wild-type GG genotype (Zheng et al., Cancer Res., 2004. 64(8):2918-2922). Within the same study set, 11 of the 17 haplotype tagSNPs examined in the TLR1-6-10 cluster were significantly associated with PCa risk estimates ranging from 1.20 (95% CI: 1.00-1.43) to 1.38 (95% CI: 11.2-1.70) comparing variant allele carriers (homozygous or heterozygous) to those with the homozygous wild-type genotype (Sun et al., J. Natl. Cancer Inst., 2005. 97(7):525-532). Both TRL4 and TLR1-6-10 were selected as possible targets because of their association with other cancers such as colon cancer and the growing theory of pathogen mediated carcinogenesis. Notably, the innate immune SNPs reported by Zheng and Sun et al. and other investigators have no apparent functional consequence. This suggests that biological validation is warranted to substantiate their impact on the innate immunity signaling pathway and PCa tumorigenesis. Moreover, despite the fact that men of African descent suffer disproportionately from PCa and may have a natural selection advantage to inheriting TLR signaling loci linked with a reduced pathogen recognition capacity (Mason et al., Prostate, 2009. 70(3):262-9), published reports on the impact of innate immunity genetic susceptibilities in this sub-group remain largely understudied.

There is one published report on the evolutionary impact of pathogen environment on alleles and haplotypes in innate immune responses and the significance in PCa susceptibility among men of African descent (Mason et al., Prostate, 2009. 70(3):262-9). The authors argue that inheritance of the CD14-260C variant allele among men of African descent is an example of antagonistic pleiotrophy, in which expression of a gene has both detrimental and beneficial effects. On the one hand, this variant allele was associated with PCa risk among African-American men. However, the authors implicate that this same loci, linked with an enhanced adaptive immune response (e.g., elevated Immunoglobulins (IgE) levels), would yield a survival advantage an environment where infection is rampant. This is partially supported in a study that revealed an association between very high concentrations IgE and a protective advantage against infection in response to helminth antigen (Hagan, Parasite Immunol, 1993. 15(1):1-4). This notion that African-Americans may have inherited mutations through natural selection in order to gain protection from persistent infection is further supported by a higher frequency of the −260CD14 variant in populations of African descent relative to European populations as well as differences in pathogen exposure in these two subgroups. Based on the frequency of the CD14 C allele and its link with a 2-fold increase in PCa, this loci allele alone may explain up to 10% of the disparity in prostate cancer incidence rates comparing African-Americans to Caucasians. However, the link between CD14 and the other twenty-four important innate targets require evaluation and validation in larger observational studies.

TABLE 1
Innate Immunity Genes

| Gene | Full Name | Chromosome | Size (kb pairs) | Marker | Alleles |
|---|---|---|---|---|---|
| TLR1 | Toll-like receptor 1 | 4q14 | 8.54 | rs4833095 | C/T |
| TLR2 | Toll-like receptor 2 | 4q32 | 21.80 | rs4696480 | T/A |
| TLR3 | Toll-like receptor 3 | 4q35 | 15.94 | rs10025405 | A/G |
| | | | | rs7657186 | G/A |
| TLR4 | Toll-like receptor 4 | 9q32-q33 | 13.31 | rs7045953 | C/T |
| | | | | rs913930 | A/G |
| | | | | rs4986790 | A/G |
| | | | | rs10759930 | T/C |
| TLR5 | Toll-like receptor 5 | 1q41-q42 | 33.04 | | |
| TLR6 | Toll-like receptor 6 | 4q14 | 2.75 | | |
| TLR7 | Toll-like receptor 7 | Xp22.3 | 23.28 | | |
| TLR8 | Toll-like receptor 8 | Xp22 | 17.10 | rs4830807 | A/C |
| | | | | rs37474 | A/G |
| TLR9 | Toll-like receptor 9 | 3p21.3 | 5.08 | | |
| TLR10 | Toll-like receptor 10 | 4q14 | 10.36 | | |
| IRAK1 | Interleukin-1 associated kinase 1 | Xq28 | 9.38 | | |
| IRAK2 | Interleukin-1 associated kinase 2 | 3p25.3 | 78.86 | rs242724 | A/C |
| | | | | rs6442161 | C/T |
| | | | | rs4684672 | G/A |
| | | | | rs708030 | C/T |
| IRAK3 | Interleukin-1 associated kinase 3 | 12q14.3 | 65.42 | | |
| IRAK4 | Interleukin-1 associated kinase 4 | 12q12 | 30.60 | rs4251545 | C/T |
| TICAM/TRIF | TIR Domain-containing adaptor molecule 1 | 19p13.3 | 15.80 | rs11672931 | A/G |
| | | | | rs6510827 | C/T |
| TOLLIP | Toll-interacting protein | 11p15.5 | 35.24 | rs5743899 | A/G |
| MyD88 | Myeloid Differentiation Primary Response | 3p22 | 4.55 | rs2239621 | T/C |
| | gene 88 | | | | |
| IRF3 | Interferon responsive factor 3 | 19q13.3 | 6.30 | rs7251 | C/G |
| IRF7 | Interferon responsive factor 7 | 11p15.5 | 12.10 | | |
| IRF8 | Interferon responsive factor 8 | 16q24.1 | 3.44 | | |
| CD14 | monocyte differentiation antigen CD14 | 5q31.1 | 1.94 | rs2569188 | A/G |
| TRAF6 | TNF receptor-assoc. factor 6 | 11q12 | 21.10 | | |
| TANK | TRAF associated NFKB activator | 2q24-q31 | 99.22 | | |
| TBK1 | TANK-binding Kinase 1 | 12q14.1 | 49.95 | | |
| BTK | Bruton agammaglobuinemia Tyrosine | 12q14.1 | 36.78 | | |
| | kinase | | | | |
| OAS1 | 2′,5′-oligoadenylate synthetase 1 | | | rs10774671 | G/A |
| RNASE1 | RNASEL ribonuclease L | | | rs3738579 | T/C |
| NFkβ1 | Nuclear Factor-kappa Beta | | | rs230528 | A/T |
| CASP9 | Caspase 9 | | | rs1052576 | G/A |
| IL-10 | Interleukin-10 | | | | |

Source: National Center for Biotechnology Institute (National Center for Biotechnology Information (NCBI) website. 2007)

### Evidence for the Impact of Inheritance of Multiple Sequence Variants and Prostate Cancer Susceptibility

It is widely accepted that complex gene interactions are implicated in prostate cancer. However, with the exception of two published reports (Sun et al., Cancer Epidemiol. Biomarkers Prev., 2006. 15(3):480-485; Xu et al., Cancer Epidemiol. Biomarkers Prev., 2005. 14(11 Pt 1):2563-2568), the previously mentioned studies concentrate on single SNP effects. In fact, Sun and co-workers (2004) evaluated interactions between the TLR 6-1-10 gene cluster and IRAK1/4 on PCa risk and observed a multiplicative interaction within the TLR1/IRAK4 axis among Swedish male participants of a case-control study (1383 PCa cases, 780 population-based controls) (Sun et al., Cancer Epidemiol. Biomarkers Prev., 2006. 15(3):480-485). In particular, men who possessed the TLR1-6399C and IRAK4 7987 had a 9.7-fold increase in PCa risk among Swedish men [OR=9.68; P=0.03; 95% confidence interval (95% CI), 1.27-73.96] relative to those with the referent genotype (Sun et al., Cancer Epidemiol. Biomarkers Prev., 2006. 15(3):480-485). These findings require subsequent validation using robust statistical tools (e.g., multi-factor dimensionality reduction (MDR)) that can evaluate complex gene interactions even in the presence of low cell counts as well as control for multiple comparisons. Recent MDR modeling efforts of Xu and co-workers (2005) revealed four-way interaction among SNPs detected in inflammatory-related SNPs detected in IL-10, IL-1RN, TIRAP and TLR5 (served as effective predictors of PCa susceptibility among Swedish men (Xu et al., Cancer Epidemiol. Biomarkers Prev., 2005. 14(11 Pt 1):2563-2568); however, these findings require replication within independent observational studies involving other Europeans and men of African descent. Intriguingly, the impact of multiple innate immunity sequence variants and their joint modifying effects on PCa risk and disease prognosis is simply unknown in men of African descent.

### Linkage Disequilibrium Analysis

Linkage disequilibrium (LD) is a measure of the degree of association between alleles in a population. One of skill in the art will appreciate that markers within one Linkage Disequilibrium Unit (LDU) of the polymorphisms described herein can also be used in a similar manner to those described herein. LDUs share an inverse relationship with LD so that regions with high LD (such as haplotype blocks) have few LDUs and low recombination, whilst regions with many LDUs have low LD and high recombination. Methods of calculating LDUs are known in the art (see, e.g., Morton et al., Proc Natl Acad Sci USA 98(9):5217-21 (2001); Tapper et al., Proc Natl Acad Sci USA 102(33):11835-11839 (2005); Maniatis et al., Proc Natl Acad Sci USA 99:2228-2233 (2002)). Thus, in some embodiments, the methods include analysis of polymorphisms that are within one LDU of a polymorphism described herein.

Alternatively, methods described herein can include analysis of polymorphisms that are within a value defined by Lewontin's D′ (linkage disequilibrium parameter, see Lewontin, Genetics 49:49-67 (1964)) of a polymorphism described herein. Results can be obtained, e.g., from on line public resources such as HapMap.org. The simple linkage disequilibrium parameter (D) reflects the degree to which alleles at two loci (for example two SNPs) occur together more often (positive values) or less often (negative values) than expected in a population as determined by the products of their respective allele frequencies. For any two loci, D can vary in value from −0.25 to +0.25. However, the magnitude of D (Dmax) varies as function of allele frequencies. To control for this, Lewontin introduced the D′ parameter, which is D/Dmax and varies in value from −1 (alleles never observed together) to +1 (alleles always observed together). Typically, the absolute value of D′ (i.e., |D′|) is reported in online databases, because it follows mathematically that positive association for one set of alleles at two loci corresponds to a negative association of equal magnitude for the reciprocal set. This disequilibrium parameter varies from 0 (no association of alleles at the two loci) to 1 (maximal possible association of alleles at the two loci).

Thus, in some embodiments, the methods include analysis of polymorphisms that are in complete linkage disequilibrium, i.e., with an R²=1 or a D′=1, for pairwise comparisons, of a polymorphism described herein.

Methods are known in the art for identifying suitable polymorphisms; for example, the International HapMap Project provides a public database that can be used, see hapmap.org, as well as The International HapMap Consortium, Nature 426:789-796 (2003), and The International HapMap Consortium, Nature 437:1299-1320 (2005). Generally, it will be desirable to use a HapMap constructed using data from individuals who share ethnicity with the subject, e.g., a HapMap for African Americans would ideally be used to identify markers within one LDU or with an R²=1 or D′=1 of a marker described herein for use in genotyping a subject of African American descent.

### Identification of Additional Markers for Use in the Methods Described Herein

In general, genetic markers can be identified using any of a number of methods well known in the art. For example, numerous polymorphisms in the regions described herein are known to exist and are available in public databases, which can be searched using methods and algorithms known in the art. Alternately, polymorphisms can be identified by sequencing either genomic DNA or cDNA in the region in which it is desired to find a polymorphism. According to one approach, primers are designed to amplify such a region, and DNA from a subject is obtained and amplified. The DNA is sequenced, and the sequence (referred to as a “subject sequence” or “test sequence”) is compared with a reference sequence, which can represent the “normal” or “wild type” sequence, or the “affected” sequence. In some embodiments, a reference sequence can be from, for example, the human draft genome sequence, publicly available in various databases, or a sequence deposited in a database such as GenBank. In some embodiments, the reference sequence is a composite of ethnically diverse individuals.

In general, if sequencing reveals a difference between the sequenced region and the reference sequence, a polymorphism has been identified. The fact that a difference in nucleotide sequence is identified at a particular site that determines that a polymorphism exists at that site. In most instances, particularly in the case of SNPs, only two polymorphic variants will exist at any location. However, in the case of SNPs, up to four variants may exist since there are four naturally occurring nucleotides in DNA. Other polymorphisms, such as insertions and deletions, may have more than four alleles.

Methods of Determining the Presence or Absence of an Allele or a Haplotype Associated with PCa or BCa

The methods described herein include determining the presence or absence of alleles or haplotypes associated with PCa or BCa. In some embodiments, an association with PCa or BCa is determined by the presence of a shared haplotype between the subject and an affected reference individual, e.g., a first or second-degree relation of the subject, and the absence of the haplotype in an unaffected reference individual. Thus the methods can include obtaining and analyzing a sample from a suitable reference individual.

Samples that are suitable for use in the methods described herein contain genetic material, e.g., genomic DNA (gDNA). Non-limiting examples of sources of samples include urine, blood, and tissue. The sample itself will typically consist of nucleated cells (e.g., blood or buccal cells), tissue, etc., removed from the subject. The subject can be an adult, child, fetus, or embryo. In some embodiments, the sample is obtained prenatally, either from a fetus or embryo or from the mother (e.g., from fetal or embryonic cells in the maternal circulation). Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

The sample may be further processed before the detecting step. For example, DNA in a cell or tissue sample can be separated from other components of the sample. The sample can be concentrated and/or purified to isolate DNA. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel et al., 2003, supra. All samples obtained from a subject, including those subjected to any sort of further processing, are considered to be obtained from the subject.

The absence or presence of a haplotype associated with PCa or BCa as described herein can be determined using methods known in the art, e.g., gel electrophoresis, capillary electrophoresis, size exclusion chromatography, sequencing, and/or arrays to detect the presence or absence of the marker(s) of the haplotype. Amplification of nucleic acids, where desirable, can be accomplished using methods known in the art, e.g., PCR.

Methods of nucleic acid analysis to detect polymorphisms and/or polymorphic variants include, e.g., microarray analysis. Hybridization methods, such as Southern analysis, Northern analysis, or in situ hybridizations, can also be used (see Current Protocols in Molecular Biology, Ausubel et al., eds., John Wiley & Sons, 2003). To detect microdeletions, fluorescence in situ hybridization (FISH) using DNA probes that are directed to a putatively deleted region in a chromosome can be used. For example, probes that detect all or a part of a microsatellite marker can be used to detect microdeletions in the region that contains that marker.

Other methods include direct manual sequencing (Church and Gilbert, Proc. Natl. Acad. Sci. USA 81:1991-1995 (1988); Sanger et al., Proc. Natl. Acad. Sci. 74:5463-5467 (1977); Beavis et al. U.S. Pat. No. 5,288,644); automated fluorescent sequencing; single-stranded conformation polymorphism assays (SSCP); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., Proc. Natl. Acad. Sci. USA 86:232-236 (1989)), mobility shift analysis (Orita et al., Proc. Natl. Acad. Sci. USA 86:2766-2770 (1989)), restriction enzyme analysis (Flavell et al., Cell 15:25 (1978); Geever et al., Proc. Natl. Acad. Sci. USA 78:5081 (1981)); quantitative real-time PCR (Raca et al., Genet Test 8(4):387-94 (2004)); heteroduplex analysis; chemical mismatch cleavage (CMC) (Cotton et al., Proc. Natl. Acad. Sci. USA 85:4397-4401 (1985)); RNase protection assays (Myers et al., Science 230:1242 (1985)); use of polypeptides that recognize nucleotide mismatches, e.g., E. coli mutS protein; allele-specific PCR, for example. See, e.g., U.S. Patent Publication No. 2004/0014095, to Gerber et al., which is incorporated herein by reference in its entirety. In some embodiments, the sequence is determined on both strands of DNA.

In order to detect polymorphisms and/or polymorphic variants, it will frequently be desirable to amplify a portion of genomic DNA (gDNA) encompassing the polymorphic site. Such regions can be amplified and isolated by PCR using oligonucleotide primers designed based on genomic and/or cDNA sequences that flank the site. See e.g., PCR Primer: A Laboratory Manual, Dieffenbach and Dveksler, (Eds.); McPherson et al., PCR Basics: From Background to Bench (Springer Verlag, 2000); Mattila et al., Nucleic Acids Res., 19:4967 (1991); Eckert et al., PCR Methods and Applications, 1:17 (1991); PCR (eds. McPherson et al., IRL Press, Oxford); and U.S. Pat. No. 4,683,202. Other amplification methods that may be employed include the ligase chain reaction (LCR) (Wu and Wallace, Genomics, 4:560 (1989), Landegren et al., Science, 241:1077 (1988), transcription amplification (Kwoh et al., Proc. Natl. Acad. Sci. USA, 86:1173 (1989)), self-sustained sequence replication (Guatelli et al., Proc. Nat. Acad. Sci. USA, 87:1874 (1990)), and nucleic acid based sequence amplification (NASBA). Guidelines for selecting primers for PCR amplification are well known in the art. See, e.g., McPherson et al., PCR Basics: From Background to Bench, Springer-Verlag, 2000. A variety of computer programs for designing primers are available, e.g., ‘Oligo’ (National Biosciences, Inc, Plymouth Minn.), MacVector (Kodak/IBI), and the GCG suite of sequence analysis programs (Genetics Computer Group, Madison, Wis. 53711).

In one example, a sample (e.g., a sample comprising genomic DNA), is obtained from a subject. The DNA in the sample is then examined to detect an allele or a haplotype as described herein. The allele or haplotype can be detected by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant.

In some embodiments, a peptide nucleic acid (PNA) probe can be used instead of a nucleic acid probe in the hybridization methods described above. PNA is a DNA mimetic with a peptide-like, inorganic backbone, e.g., N-(2-aminoethyl)glycine units, with an organic base (A, G, C, T or U) attached to the glycine nitrogen via a methylene carbonyl linker (see, e.g., Nielsen et al., Bioconjugate Chemistry, The American Chemical Society, 5:1 (1994)). The PNA probe can be designed to specifically hybridize to a nucleic acid comprising a polymorphic variant conferring susceptibility to or indicative of the presence of PCa or BCa.

In some embodiments, restriction digest analysis can be used to detect the existence of a polymorphic variant of a polymorphism, if alternate polymorphic variants of the polymorphism result in the creation or elimination of a restriction site. A sample containing genomic DNA is obtained from the individual. Polymerase chain reaction (PCR) can be used to amplify a region comprising the polymorphic site, and restriction fragment length polymorphism analysis is conducted (see Ausubel et al., Current Protocols in Molecular Biology, supra). The digestion pattern of the relevant DNA fragment indicates the presence or absence of a particular polymorphic variant of the polymorphism and is therefore indicative of the presence or absence of susceptibility to PCa or BCa.

Sequence analysis can also be used to detect specific polymorphic variants. A sample comprising DNA or RNA is obtained from the subject. PCR or other appropriate methods can be used to amplify a portion encompassing the polymorphic site, if desired. The sequence is then ascertained, using any standard method, and the presence of a polymorphic variant is determined.

Allele-specific oligonucleotides can also be used to detect the presence of a polymorphic variant, e.g., through the use of dot-blot hybridization of amplified oligonucleotides with allele-specific oligonucleotide (ASO) probes (see, for example, Saiki et al., Nature (London) 324:163-166 (1986)). An “allele-specific oligonucleotide” (also referred to herein as an “allele-specific oligonucleotide probe”) is typically an oligonucleotide of approximately 10-50 base pairs, preferably approximately 15-30 base pairs, that specifically hybridizes to a nucleic acid region that contains a polymorphism. An allele-specific oligonucleotide probe that is specific for particular a polymorphism can be prepared using standard methods (see Ausubel et al., Current Protocols in Molecular Biology, supra).

Generally, to determine which of multiple polymorphic variants is present in a subject, a sample comprising DNA is obtained from the individual. PCR can be used to amplify a portion encompassing the polymorphic site. DNA containing the amplified portion may be dot-blotted, using standard methods (see Ausubel et al., Current Protocols in Molecular Biology, supra), and the blot contacted with the oligonucleotide probe. The presence of specific hybridization of the probe to the DNA is then detected. Specific hybridization of an allele-specific oligonucleotide probe (specific for a polymorphic variant indicative of susceptibility to PCa or BCa) to DNA from the subject is indicative of susceptibility to PCa or BCa.

In some embodiments, fluorescence polarization template-directed dye-terminator incorporation (FP-TDI) is used to determine which of multiple polymorphic variants of a polymorphism is present in a subject (Chen et al., (1999) Genome Research, 9(5):492-498). Rather than involving use of allele-specific probes or primers, this method employs primers that terminate adjacent to a polymorphic site, so that extension of the primer by a single nucleotide results in incorporation of a nucleotide complementary to the polymorphic variant at the polymorphic site.

Real-time pyrophosphate DNA sequencing is yet another approach to detection of polymorphisms and polymorphic variants (Alderborn et al., (2000) Genome Research, 10(8):1249-1258). Additional methods include, for example, PCR amplification in combination with denaturing high performance liquid chromatography (dHPLC) (Underhill, P. A., et al., Genome Research, Vol. 7, No. 10, pp. 996-1005, 1997).

The methods can include determining the genotype of a subject with respect to both copies of the polymorphic site present in the genome. For example, the complete genotype may be characterized as −/−, as −/+, or as +/+, where a minus sign indicates the presence of the reference or wild type sequence at the polymorphic site, and the plus sign indicates the presence of a polymorphic variant other than the reference sequence. If multiple polymorphic variants exist at a site, this can be appropriately indicated by specifying which ones are present in the subject. Any of the detection means described herein can be used to determine the genotype of a subject with respect to one or both copies of the polymorphism present in the subject's genome.

In some embodiments, it is desirable to employ methods that can detect the presence of multiple polymorphisms (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple polymorphic variants (e.g., polymorphic variants at a plurality of polymorphic sites) in parallel or substantially simultaneously according to certain embodiments of the invention.

### Probes

Nucleic acid probes can be used to detect and/or quantify the presence of a particular target nucleic acid sequence within a sample of nucleic acid sequences, e.g., as hybridization probes, or to amplify a particular target sequence within a sample, e.g., as a primer. Probes have a complimentary nucleic acid sequence that selectively hybridizes to the target nucleic acid sequence. In order for a probe to hybridize to a target sequence, the hybridization probe must have sufficient identity with the target sequence, i.e., at least 70%, e.g., 80%, 90%, 95%, 98% or more identity to the target sequence. The probe sequence must also be sufficiently long so that the probe exhibits selectivity for the target sequence over non-target sequences. For example, the probe will be at least 20, e.g., 25, 30, 35, 50, 100, 200, 300, 400, 500, 600, 700, 800, 900 or more, nucleotides in length. In some embodiments, the probes are not more than 30, 50, 100, 200, 300, 500, 750, or 1000 nucleotides in length. Probes are typically about 20 to about 1×10⁶ nucleotides in length. Probes include primers, which generally refers to a single-stranded oligonucleotide probe that can act as a point of initiation of template-directed DNA synthesis using methods such as PCR (polymerase chain reaction), LCR (ligase chain reaction), etc., for amplification of a target sequence. In some embodiments, the probe is a test probe, e.g., a probe that can be used to detect polymorphisms in a region described herein, e.g., polymorphisms as described herein. In some embodiments, the probe can bind to another marker sequence associated with PCa or BCa, as described herein.

Control probes can also be used. For example, a probe that binds a less variable sequence, e.g., repetitive DNA associated with a centromere of a chromosome, can be used as a control. Probes that hybridize with various centromeric DNA and locus-specific DNA are available commercially, for example, from Vysis, Inc. (Downers Grove, Ill.), Molecular Probes, Inc. (Eugene, Oreg.), or from Cytocell (Oxfordshire, UK). Probe sets are available commercially, e.g., from Applied Biosystems, e.g., the Assays-on-Demand SNP kits. Alternatively, probes can be synthesized, e.g., chemically or in vitro, or made from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via the polymerase chain reaction (PCR). See, for example, Nath and Johnson, Biotechnic. Histochem., 1998, 73(1):6-22, Wheeless et al., Cytometry 1994, 17:319-326, and U.S. Pat. No. 5,491,224.

In some embodiments, the probes are labeled, e.g., by direct labeling, with a fluorophore, an organic molecule that fluoresces after absorbing light of lower wavelength/higher energy. A directly labeled fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, e.g., U.S. Pat. No. 5,491,224.

Fluorophores of different colors can be chosen such that each probe in a set can be distinctly visualized. For example, a combination of the following fluorophores can be used: 7-amino-4-methylcoumarin-3-acetic acid (AMCA), Texas Red™ (Molecular Probes, Inc., Eugene, Oreg.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and Cascade™ blue acetylazide (Molecular Probes, Inc., Eugene, Oreg.). Fluorescently labeled probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine the hybridization pattern of the probes. Fluorescence-based arrays are also known in the art.

In other embodiments, the probes can be indirectly labeled with, e.g., biotin or digoxygenin, or labeled with radioactive isotopes such as ³²P and ³H. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Oligonucleotide probes that exhibit differential or selective binding to polymorphic sites may readily be designed by one of ordinary skill in the art. For example, an oligonucleotide that is perfectly complementary to a sequence that encompasses a polymorphic site (i.e., a sequence that includes the polymorphic site, within it or at one end) will generally hybridize preferentially to a nucleic acid comprising that sequence, as opposed to a nucleic acid comprising an alternate polymorphic variant.

### Arrays and Uses Thereof

In another aspect, the invention features arrays that include a substrate having a plurality of addressable areas, and methods of using them. At least one area of the plurality includes a nucleic acid probe that binds specifically to a sequence comprising a polymorphism listed in Table 1, and can be used to detect the absence or presence of said polymorphism, e.g., one or more SNPs, microsatellites, minisatellites, or indels, as described herein, to determine a haplotype. For example, the array can include one or more nucleic acid probes that can be used to detect a polymorphism listed in Table 1. In some embodiments, the array further includes at least one area that includes a nucleic acid probe that can be used to specifically detect another marker associated with PCa or BCa, as described herein. The substrate can be, e.g., a two-dimensional substrate known in the art such as a glass slide, a wafer (e.g., silica or plastic), a mass spectroscopy plate, or a three-dimensional substrate such as a gel pad. In some embodiments, the probes are nucleic acid capture probes.

Methods for generating arrays are known in the art and include, e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145). The array typically includes oligonucleotide probes capable of specifically hybridizing to different polymorphic variants. According to the method, a nucleic acid of interest, e.g., a nucleic acid encompassing a polymorphic site, (which is typically amplified) is hybridized with the array and scanned. Hybridization and scanning are generally carried out according to standard methods. See, e.g., Published PCT Application Nos. WO 92/10092 and WO 95/11995, and U.S. Pat. No. 5,424,186. After hybridization and washing, the array is scanned to determine the position on the array to which the nucleic acid hybridizes. The hybridization data obtained from the scan is typically in the form of fluorescence intensities as a function of location on the array.

Arrays can include multiple detection blocks (i.e., multiple groups of probes designed for detection of particular polymorphisms). Such arrays can be used to analyze multiple different polymorphisms. Detection blocks may be grouped within a single array or in multiple, separate arrays so that varying conditions (e.g., conditions optimized for particular polymorphisms) may be used during the hybridization. For example, it may be desirable to provide for the detection of those polymorphisms that fall within G-C rich stretches of a genomic sequence, separately from those falling in A-T rich segments.

Additional description of use of oligonucleotide arrays for detection of polymorphisms can be found, for example, in U.S. Pat. Nos. 5,858,659 and 5,837,832. In addition to oligonucleotide arrays, cDNA arrays may be used similarly in certain embodiments of the invention.

The methods described herein can include providing an array as described herein; contacting the array with a sample, e.g., a portion of genomic DNA that includes at least one marker described herein or another chromosome, e.g., including another region or marker associated with PCa or BCa, and detecting binding of a nucleic acid from the sample to the array. Optionally, the method includes amplifying nucleic acid from the sample, e.g., genomic DNA that includes a portion of a human chromosome described herein, and, optionally, a region that includes another region associated with PCa or BCa, prior to or during contact with the array.

In some aspects, the methods described herein can include using an array that can ascertain differential expression patterns or copy numbers of one or more genes in samples from normal and affected individuals (see, e.g., Redon et al., Nature. 444(7118):444-54 (2006)). For example, arrays of probes to a marker described herein can be used to measure polymorphisms between DNA from a subject having PCa or BCa, and control DNA, e.g., DNA obtained from an individual that does not have PCa or BCa, and has no risk factors for PCa or BCa. Since the clones on the array contain sequence tags, their positions on the array are accurately known relative to the genomic sequence. Different hybridization patterns between DNA from an individual afflicted with PCa or BCa and DNA from a normal individual at areas in the array corresponding to markers as described herein, and, optionally, one or more other regions associated with PCa or BCa, are indicative of a risk of PCa or BCa. Methods for array production, hybridization, and analysis are described, e.g., in Snijders et al., (2001) Nat. Genetics 29:263-264; Klein et al., (1999) Proc. Natl. Acad. Sci. U.S.A. 96:4494-4499; Albertson et al., (2003) Breast Cancer Research and Treatment 78:289-298; and Snijders et al. “BAC microarray based comparative genomic hybridization.” In: Zhao et al. (Eds.), Bacterial Artificial Chromosomes: Methods and Protocols, Methods in Molecular Biology, Humana Press, 2002. Real time quantitative PCR can also be used to determine copy number.

In another aspect, the invention features methods of determining the absence or presence of an allele or a haplotype associated with PCa or BCa as described herein, using an array described above. The methods include providing a two dimensional array having a plurality of addresses, each address of the plurality being positionally distinguishable from each other address of the plurality having a unique nucleic acid capture probe, contacting the array with a first sample from a test subject who is suspected of having or being at risk for PCa or BCa, and comparing the binding of the first sample with one or more references, e.g., binding of a sample from a subject who is known to have PCa or BCa, and/or binding of a sample from a subject who is unaffected, e.g., a control sample from a subject who neither has, nor has any risk factors for PCa or BCa. In some embodiments, the methods include contacting the array with a second sample from a subject who has PCa or BCa; and comparing the binding of the first sample with the binding of the second sample. In some embodiments, the methods include contacting the array with a third sample from a cell or subject that does not have PCa or BCa and is not at risk for PCa or BCa; and comparing the binding of the first sample with the binding of the third sample. In some embodiments, the second and third samples are from first or second-degree relatives of the test subject. Binding, e.g., in the case of a nucleic acid hybridization, with a capture probe at an address of the plurality, can be detected by any method known in the art, e.g., by detection of a signal generated from a label attached to the nucleic acid.

### Kits

Also within the scope of the invention are kits comprising a probe that hybridizes with a region of human chromosome as described herein and can be used to detect a polymorphism described herein. The kit can include one or more other elements including: instructions for use; and other reagents, e.g., a label, or an agent useful for attaching a label to the probe. Instructions for use can include instructions for diagnostic applications of the probe for assessing risk of PCa or BCa in a method described herein. Other instructions can include instructions for attaching a label to the probe, instructions for performing in situ analysis with the probe, and/or instructions for obtaining a sample to be analyzed from a subject. As discussed above, the kit can include a label, e.g., any of the labels described herein. In some embodiments, the kit includes a labeled probe that hybridizes to a region of human chromosome as described herein, e.g., a labeled probe as described herein.

The kit can also include one or more additional probes that hybridize to the same chromosome or another chromosome or portion thereof that can have an abnormality associated with risk for PCa or BCa. For example, the additional probe or probes can be a probe that hybridizes to a marker described herein, or rs6983267, rs1447295, rs4430796, rs7501939, rs3760511, rs1859962, rs7214479, rs6501455, rs983085, rs6983561, rs16901979, rs6983267, rs7000448, rs1447295, rs4242382, rs7017300, rs10090154, and rs7837688 (Zheng et al., NEJM 358:910-919 (2008)). A kit that includes additional probes can further include labels, e.g., one or more of the same or different labels for the probes. In other embodiments, the additional probe or probes provided with the kit can be a labeled probe or probes. When the kit further includes one or more additional probe or probes, the kit can further provide instructions for the use of the additional probe or probes.

Kits for use in self-testing can also be provided. For example, such test kits can include devices and instructions that a subject can use to obtain a sample, e.g., of buccal cells or blood, without the aid of a health care provider. For example, buccal cells can be obtained using a buccal swab or brush, or using mouthwash.

Kits as provided herein can also include a mailer, e.g., a postage paid envelope or mailing pack, that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms, e.g., the test requisition form, and the container holding the sample, can be coded, e.g., with a bar code, for identifying the subject who provided the sample.

### Databases

Also provided herein are databases that include a list of polymorphisms as described herein, and wherein the list is largely or entirely limited to polymorphisms identified as useful in performing genetic diagnosis of or determination of susceptibility to PCa or BCa as described herein. The list is stored, e.g., on a flat file or computer-readable medium. The databases can further include information regarding one or more subjects, e.g., whether a subject is affected or unaffected, clinical information such as age of onset of symptoms, any treatments administered and outcomes (e.g., data relevant to pharmacogenomics, diagnostics, or theranostics), and other details, e.g., about the disorder in the subject, or environmental or other genetic factors. The databases can be used to detect correlations between a particular haplotype and the information regarding the subject, e.g., to detect correlations between a haplotype and a particular phenotype, or treatment response.

### Engineered Cells

Also provided herein are engineered cells that harbor one or more polymorphism described herein, e.g., one or more polymorphisms that constitute a haplotype associated with PCa or BCa. Such cells are useful for studying the effect of a polymorphism on physiological function, and for identifying and/or evaluating potential therapeutic agents for the treatment of PCa or BCa, e.g., anti-cancer drugs.

As one example, included herein are cells in which one of the various alleles of the genes described herein has been re-created that is associated with an increased risk of PCa or BCa. Methods are known in the art for generating cells, e.g., by homologous recombination between the endogenous gene and an exogenous DNA molecule introduced into a cell, e.g., a cell of an animal. In some embodiments, the cells can be used to generate transgenic animals using methods known in the art.

The cells are preferably mammalian cells, e.g., epithelial or endothelial type cells, in which an endogenous gene has been altered to include a polymorphism as described herein. Cells can be BCa cell lines (e.g., 1143, 1806, 231, 231ALPHA, MDA-MB-435, MDA-MB-468, MCF7, ZR-751) or PCa cell lines (e.g., LnCap, MCF7, ZR-751, 22RV11). Techniques such as targeted homologous recombinations, can be used to insert the heterologous DNA as described in, e.g., Chappel, U.S. Pat. No. 5,272,071; WO 91/06667, published in May 16, 1991.

The invention is further described in the following examples, which do not limit the scope of the invention described in the claims.

### EXAMPLES

The invention is further described in the following examples, which do not limit the scope of the invention described in the claims.

### Example 1

### Quality Assurance of TaqMan Allelic Discrimination Technique

A high-throughput 48-plex Beckman SNPstream allelic discrimination method was used to discriminate among twenty-six innate immunity alleles among 307 men of African descent. Nineteen SNPs were successfully genotyped using the SNPstream platform, as summarized in Table 2. For each batch analysis (n=96), eight germ-line DNA samples were genotyped in duplicate to estimate the percent concordance between repeat samples. The percent concordance among duplicate samples for the aforementioned SNPs ranged was 100%. Given the high percent agreement within the repeat genotype analysis, the ability to accurately perform SNP analysis using the SNPstream platform was demonstrated. With the exception of two loci (Tollip_rs5743899 and IRFS rs2004640), the genotype failure rate was negligible (i.e., ≦3.7%).

TABLE 2
Association between Prostate Cancer Risk and Innate Immunity
Sequence Variants among Men of African Descent

| SNP | Cases (%) | Ctrl (%) | OR (95% CI) | OR (95% CI)adj | P-value | P-trend |
|---|---|---|---|---|---|---|
| CD14_2569188 | | | | | | |
| AA | 57 (33.7) | 46 (33.3) | 1.00 | 1.00 | 0.782 | 0.673 |
| AG | 84 (49.7) | 65 (47.1) | 1.04 (0.63, 1.73) | 0.73 (0.40, 1.36) | | |
| GG | 28 (16.6) | 27 (19.6) | 0.84 (0.43, 1.61) | 0.63 (0.28, 1.43) | | |
| AG + GG | 112 (66.3) | 92 (66.7) | 0.98 (0.61, 1.58) | 0.71 (0.39, 1.27) | | |
| TOLLIP_5743899 | | | | | | |
| AA | 42 (29.6) | 48 (42.9) | 1.00 | 1.00 | 0.002 | 0.562 |
| AG | 88 (62.0) | 45 (40.2) | 2.24 (1.29, 3.87) | 2.69 (1.38, 5.27) | | |
| GG | 12 (8.4) | 19 (16.9) | 0.72 (0.31, 1.66) | 0.91 (0.33, 2.51) | | |
| AG + GG | 100 (70.4) | 64 (57.1) | 1.79 (1.06, 3.00) | 2.17 (1.14, 4.12) | | |
| TICAM_11672931 | | | | | | |
| AA | 79 (47.3) | 62 (46.3) | 1.00 | 1.00 | 0.809 | 0.658 |
| AG | 52 (31.1) | 39 (29.1) | 1.05 (0.62, 1.78) | 1.27 (0.66, 2.43) | | |
| GG | 36 (21.6) | 33 (24.6) | 0.86 (0.48, 1.53) | 0.80 (0.40, 1.63) | | |
| AG + GG | 88 (52.7) | 72 (53.7) | 0.96 (0.61, 1.51) | 1.04 (0.60, 1.81) | | |
| TLR4_7045953 | | | | | | |
| TT | 89 (52.4) | 72 (51.8) | 1.00 | 1.00 | 0.169 | 0.482 |
| CT | 60 (35.3) | 58 (41.7) | 0.84 (0.52, 1.35) | 1.18 (0.66, 2.11) | | |
| CC | 21 (12.3) | 9 (6.5) | 1.89 (0.81, 4.37) | 2.15 (0.78, 5.91) | | |
| CT + CC | 81 (47.6) | 67 (48.2) | 0.98 (0.62, 1.53) | 1.32 (0.76, 2.30) | | |
| TLR1_4833095 | | | | | | |
| CC | 116 (69.0) | 84 (60.4) | 1.00 | 1.00 | 0.181 | 0.071 |
| CT | 46 (27.4) | 45 (32.4) | 0.74 (0.45, 1.22) | 0.67 (0.37, 1.23) | | |
| TT | 6 (3.6) | 10 (7.2) | 0.43 (0.15, 1.24) | 0.34 (0.07, 1.57) | | |
| CT + TT | 52 (31.0) | 55 (39.6) | 0.68 (0.43, 1.10) | 0.62 (0.35, 1.12) | | |
| IRAK2_242724 | | | | | | |
| AA | 104 (62.3) | 97 (68.8) | 1.00 | 1.00 | 0.323 | 0.387 |
| AC | 59 (35.3) | 39 (27.7) | 1.41 (0.86, 2.30) | 1.20 (0.67, 2.18) | | |
| CC | 4 (2.4) | 5 (3.5) | 0.75 (0.19, 2.86) | 0.83 (0.13, 5.22) | | |
| AC + CC | 63 (37.7) | 44 (31.2) | 1.33 (0.83, 2.15) | 1.17 (0.66, 2.09) | | |
| TLR2_4696480 | | | | | | |
| TT | 65 (38.5) | 61 (43.6) | 1.00 | 1.00 | 0.131 | 0.097 |
| TA | 66 (39.0) | 60 (42.9) | 1.03 (0.63, 1.69) | 1.33 (0.72, 2.47) | | |
| AA | 38 (22.5) | 19 (13.5) | 1.88 (0.98, 3.60) | 2.60 (1.18, 5.71) | | |
| TA + AA | 104 (61.5) | 79 (56.4) | 1.24 (0.78, 1.95) | 1.64 (0.93, 2.90) | | |
| IRAK4_4251545 | | | | | | |
| CC | 95 (55.9) | 69 (48.9) | 1.00 | 1.00 | 0.448 | 0.326 |
| CT | 62 (36.5) | 61 (43.3) | 0.74 (0.46, 1.18) | 0.64 (0.36, 1.16) | | |
| TT | 13 (7.6) | 11 (7.8) | 0.86 (0.36, 2.03) | 0.90 (0.31, 2.62) | | |
| CT + TT | 75 (44.1) | 72 (51.1) | 0.76 (0.48, 1.18) | 0.68 (0.39, 1.19) | | |
| OAS1_10774671 | | | | | | |
| GG | 48 (27.9) | 46 (32.9) | 1.00 | 1.00 | 0.556 | 0.280 |
| GA | 87 (50.6) | 69 (49.3) | 1.21 (0.72, 2.02) | 0.99 (0.54, 1.84) | | |
| AA | 37 (21.5) | 25 (17.8) | 1.42 (0.74, 2.71) | 0.87 (0.40, 1.92) | | |
| GA + AA | 124 (72.1) | 94 (67.1) | 1.26 (0.78, 2.05) | 0.96 (0.54, 1.71) | | |
| IRAK2_6442161 | | | | | | |
| CC | 83 (49.4) | 77 (55.8) | 1.00 | 1.00 | 0.335 | 0.543 |
| CT | 71 (42.3) | 47 (34.1) | 1.40 (0.87-2.27) | 1.05 (0.59-1.87) | | |
| TT | 14 (8.3) | 14 (10.1) | 0.93 (0.42-2.07) | 0.88 (0.32-2.40) | | |
| CT + TT | 85 (50.6) | 61 (44.2) | 1.29 (0.82-2.03) | 1.01 (0.59-1.75) | | |
| IRF3_7251 | | | | | | |
| CC | 64 (37.6) | 50 (36.8) | 1.00 | 1.00 | 0.630 | 0.785 |
| CG | 90 (52.9) | 77 (56.6) | 0.91 (0.57-1.47) | 1.07 (0.60-1.91) | | |
| GG | 16 (9.4) | 9 (6.6) | 1.39 (0.57-3.40) | 1.69 (0.58-4.95) | | |
| CG + GG | 106 (62.3) | 86 (63.2) | 0.96 (0.60-1.54) | 1.13 (0.64-2.00) | | |
| MYD88_2239621 | | | | | | |
| TT | 61 (35.7) | 44 (31.2) | 1.00 | 1.00 | 0.434 | 0.842 |
| TC | 76 (44.4) | 73 (51.8) | 0.75 (0.45-1.24) | 0.72 (0.39-1.33) | | |
| CC | 34 (19.9) | 24 (17.0) | 1.02 (0.53-1.96) | 0.82 (0.36-1.87) | | |
| TC + CC | 110 (64.3) | 97 (68.8) | 0.82 (0.51-1.31) | 0.75 (0.42-1.33) | | |
| IRAK2_4684672 | | | | | | |
| GG | 107 (62.9) | 90 (64.3) | 1.00 | 1.00 | 0.640 | 0.583 |
| GA | 53 (31.2) | 45 (32.1) | 0.99 (0.61-1.61) | 0.97 (0.54-1.77) | | |
| AA | 10 (5.9) | 5 (3.6) | 1.68 (0.55-5.10) | 1.13 (0.31-4.17) | | |
| GA + AA | 63 (35.7) | 50 (35.7) | 1.06 (0.67-1.69) | 0.99 (0.56-1.76) | | |
| TLR3_10025405 | | | | | | |
| AA | 102 (59.6) | 75 (53.2) | 1.00 | 1.00 | 0.318 | 0.513 |
| AG | 54 (31.6) | 56 (39.7) | 0.71 (0.44-1.14) | 0.86 (0.47-1.55) | | |
| GG | 15 (8.8) | 10 (7.1) | 1.10 (0.47-2.59) | 1.83 (0.60-5.61) | | |
| AG + GG | 69 (40.3) | 66 (46.8) | 0.77 (0.49-1.21) | 0.97 (0.56-1.70) | | |
| RNASE1_3738579 | | | | | | |
| TT | 125 (72.7) | 104 (74.8) | 1.00 | 1.00 | 0.700 | 0.559 |
| TC | 44 (25.6) | 34 (24.5) | 1.08 (0.64-1.81) | 0.93 (0.50-1.75) | | |
| CC | 3 (1.7) | 1 (0.7) | 2.50 (0.26-24.26) | >999 (<0.00 > 999) | | |
| TC + CC | 47 (27.3) | 35 (25.2) | 1.12 (0.67-1.86) | 1.02 (0.55-1.89) | | |
| TLR4_913930 | | | | | | |
| AA | 130 (76.5) | 104 (74.8) | 1.00 | 1.00 | 0.921 | 0.695 |
| AG | 35 (20.6) | 30 (21.6) | 0.93 (0.54-1.62) | 1.01 (0.51-1.98) | | |
| GG | 5 (2.9) | 5 (3.6) | 0.80 (0.23-2.84) | 063 (0.13-2.95) | | |
| AG + GG | 40 (23.5) | 35 (25.2) | 0.91 (0.54-1.54) | 0.94 (0.50-1.78) | | |
| TLR3_7657186 | | | | | | |
| GG | 91 (53.5) | 79 (57.2) | 1.00 | 1.00 | 0.402 | 0.299 |
| GA | 67 (39.4) | 54 (39.1) | 1.08 (0.67-1.72) | 1.25 (0.70-2.24) | | |
| AA | 12 (7.1) | 5 (3.6) | 2.08 (0.70-6.17) | 1.41 (0.42-4.76) | | |
| GA + AA | 79 (46.5) | 59 (42.7) | 1.16 (0.74-1.83) | 1.27 (0.73-2.22) | | |
| TLR4_4986790 | | | | | | |
| AA | 147 (85.5) | 121 (85.8) | 1.00 | 1.00 | 0.712 | 0.759 |
| AG | 22 (12.8) | 19 (13.5) | 0.95 (0.49-1.84) | 0.93 (0.40-2.19) | | |
| GG | 3 (1.7) | 1 (0.7) | 2.47 (0.25-24.04) | 2.05 (0.12-33.82) | | |
| AG + GG | 25 (14.5) | 20 (14.2) | 1.03 (0.54-1.94) | 0.99 (0.43-2.26) | | |
| TLR6_5743808 | | | | | | |
| TT | 127 (69) | 239 (80) | 1.00 | | 0.004 | |
| TC | 58 (31) | 58 (19) | | | | |
| CC | 0 (0) | 1 (1) | | | | |
| TC + CC | 58 (31) | 59 (20) | 1.85 (1.21-2.82) | | | |

^{†} Associations were determined using multivariate LR models to estimate the risk of developing PCa using as the reference genotypes.
^{††} Risk estimates adjusted for age (continuous variable) and prostate specific antigen (continuous variable).
^{†††} Differences in the frequency of variant and referent genotypes between cases and controls were determined using the chi-square test of association and a significance level of 0.05.

### Example 2

### Prevalence of Innate Immunity-Related Alleles Among Men of African Descent

Significant progress has been made on the analysis of nineteen sequence variants within fourteen innate immunity-associated genes among 307 men of African Descent (169 prostate cancer cases and 138 controls). In this study population, the minor allele frequencies for 19 innate immunity signalling SNPs (>5%) were fairly common and consistent with other men of African Descent as reported by the National Center for Biotechnology Institute (National Center for Biotechnology Information (NCBI) website. 2007). The innate immunity genotype frequencies among controls did not differ from expected distributions based upon the Hardy-Weinberg equilibrium and the chi-square test of heterogeneity (P>0.03) based on a significance cut-off value of 0.005.

### Example 3

### Single Gene Effects in Relation to Prostate Cancer Among Men of African Descent

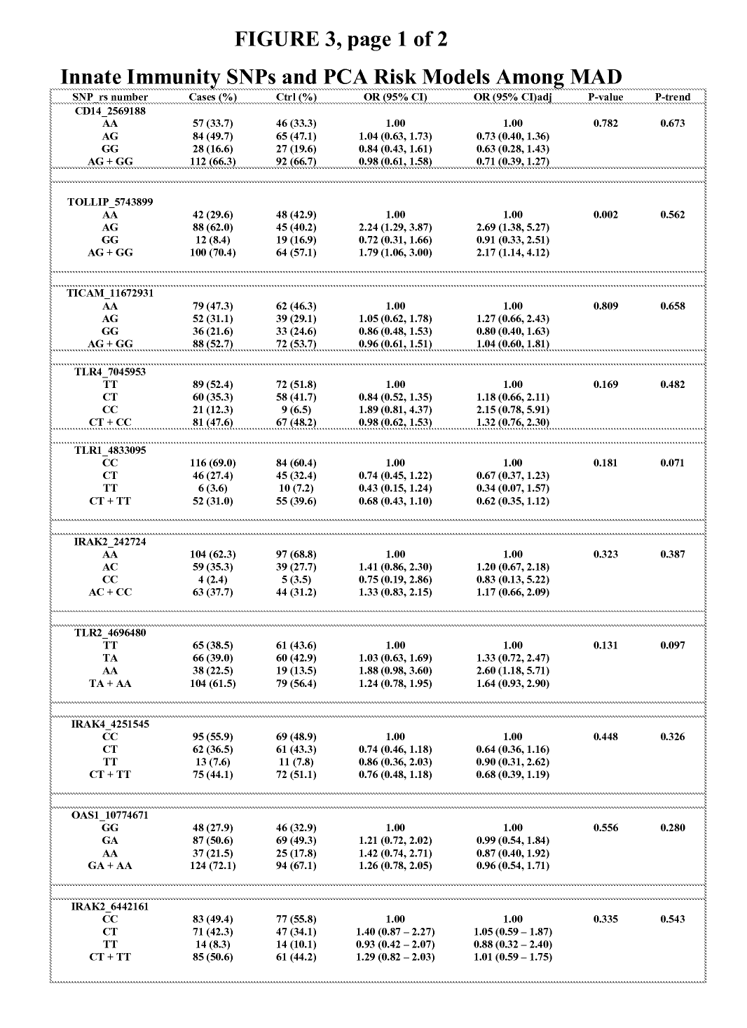

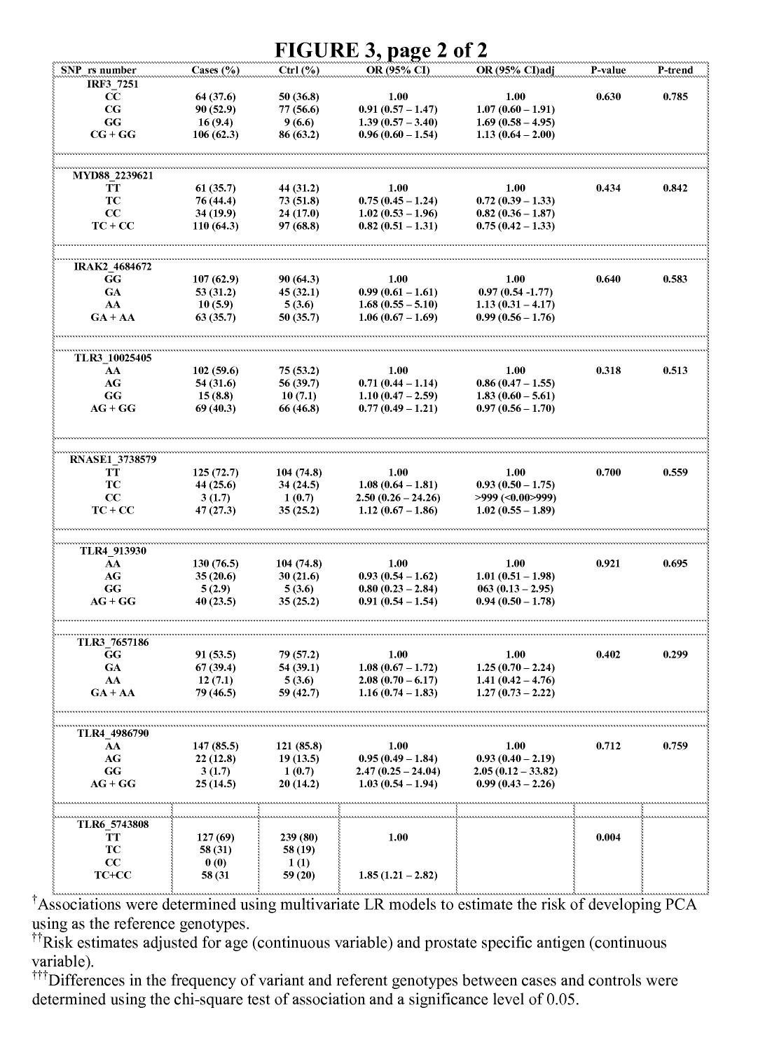

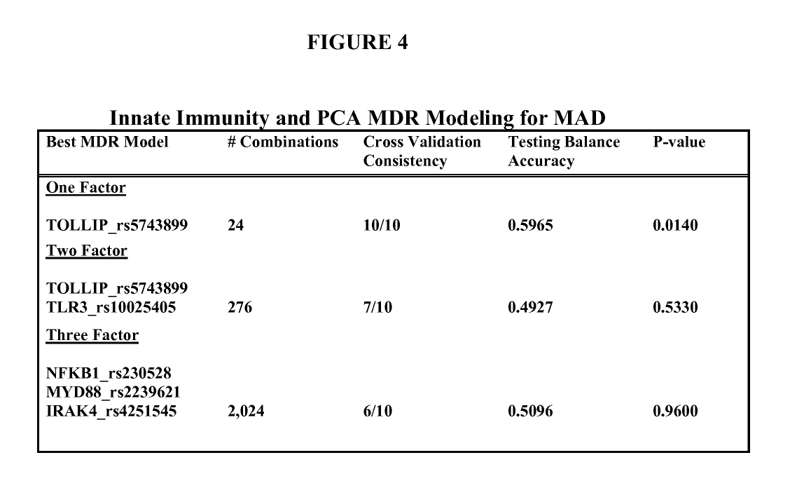

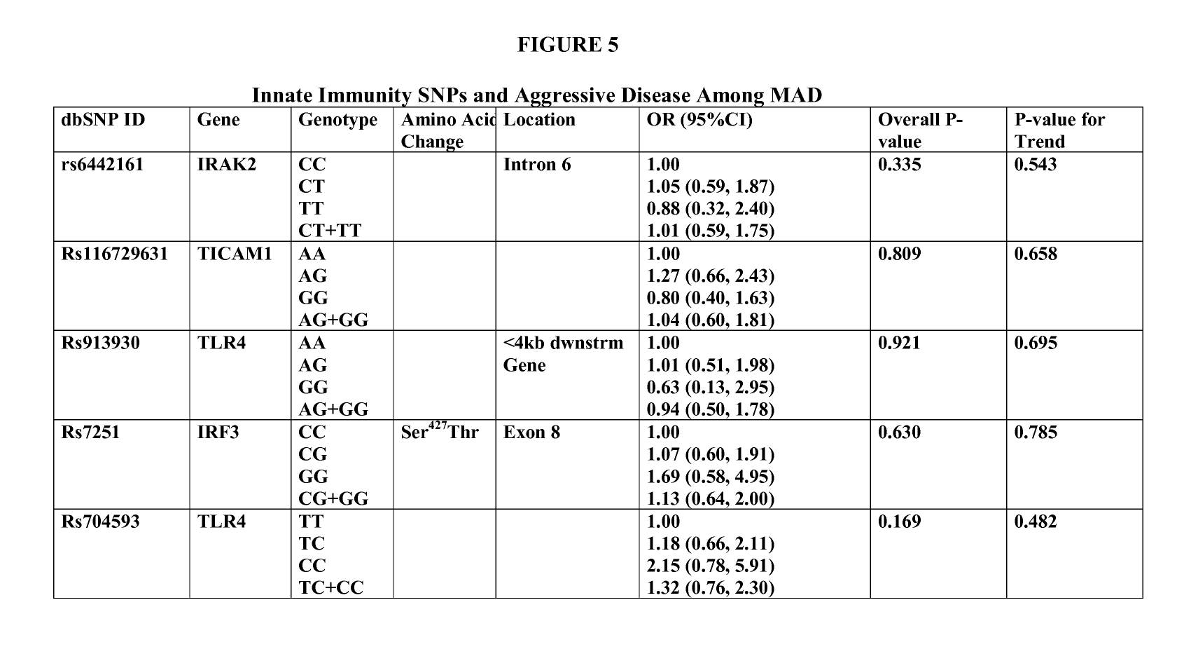

The independent effects of 19 variant innate immunity alleles were evaluated in relation to PCa susceptibility among men of African descent. For this analysis 169 men diagnosed with prostate cancer and 138 controls were genotyped for nineteen SNPs detected in innate immunity genes (e.g., TLR 1-4, CD14, IRAK2, IRAK4, IRF3, IRFS, MYD88, OAS1, TICAM1, TOLLIP, RNAseL) using a SNPstream genotyping platform. Multiple coding and non-coding (i.e., promoter, 3′UTR, 5′UTR, and intronic) SNPs spanning each gene were chosen from in silico databases and published reports using one or more of the following criteria: a minor allele frequency >5%; location within the gene; disparities in the allele frequencies comparing men of European and African descent, and empirical evidence supporting their role in cancer malignancies or other chronic diseases. One hypothesis was that individuals harboring innate immunity loci linked with enhanced pro-inflammatory response may modify PCa susceptibility and disease progression. Main effects in relation to prostate cancer risk were assessed using conventional logistic regression analysis. Although none of the markers were associated with high tumor grade, three out of nineteen innate immunity related loci detected in TOLLIP rs5743899G (overall chi-square p-value=0.002; 1000-fold permutation testing p-value=0.01), TLR6_{—}5743808TT+CC (P=0.004; bonferroni corrected p-value=0.076) and the TLR2 promoter region (rs4696480AA) resulted in a 1.8-2.6 fold increase in relation to prostate cancer susceptibility, as summarized in FIGS. 3-5. Notably, the TLR2 “A” allele at position −16934 is linked with enhanced cytokine production (i.e., interleukin (IL)-6, IL-12, and tumor necrosis factor-alpha (TNF-α) (Veltkamp et al., Clin Exp Immunol, 2007. 149(3):453-62). This pro-inflammatory response may ultimately promote tumor growth and metastasis; however, this remains to be demonstrated in PCa cell lines. These findings suggest that genetic alterations detected within the innate immunity signaling pathways modify prostate cancer susceptibility.

### Example 4

### Study Population: Resources for Men of African And European Descent

Study participants from Men of African Descent Prostate Cancer (MADCaP) Consortium and the African Caribbean Cancer Consortium (AC3) are included in the study based on the willingness of subjects to participate in ancillary studies as well as availability of consent forms that permit sharing of de-identified data with other investigators, germ-line DNA, genotype data, and clinico-pathological data.

The MADCaP Consortium consists of 18 observational studies from various domestic and international academic institutions involving de-identified germ-line DNA samples, genotype data, as well as: (i) patient characteristics (age at diagnosis/ascertainment, family history of PCa in fist degree relatives, body mass index, smoking history (age at starting, age at quitting, number of cig/day, other type of tobacco used, exposure to environmental tobacco smoke, country of residence, Global West African (0-100%)); (ii) clinico-pathological data (e.g., tumor stage/grade/size, androgen receptor status, following diagnosis, PSA); (iii) treatment (chemo-/hormone/radiation treatment status); and (iv) prognostic outcomes (e.g., PSA relapse, short time from treatment Nadir, time from Nadir to androgen independent PCa, PSA progression free survival, disease-free survival, progression-free survival, overall survival) from over 10,753 African-American and 45,557 European PCa cases and controls.

### Example 5

### Howard University Prostate Cancer Study (HUPCS)

Unrelated male residents (n=1016) of Washington, D.C. and Columbia, S.C., were considered for eligibility in the current PCa case control study. Study participants (n=132) were not considered in the current study if they met one or more of the following exclusion criteria: (1) they were diagnosed with benign prostatic hyperplasia (n=64); (2) had an abnormal PSA and DRE (n=11); and (3) had European ancestry based on a Global Ancestry score of <25% (n=70) (Sun et al., J. Natl. Cancer Inst., 2005. 97(7):525-532). Eligible men of African descent (i.e., self-identified African-Americans, East Africans-Americans, West Africans-Americans, and Afro-Caribbeans), including 208 patients (ages 41-91) and 665 healthy volunteers (ages 26-89), were recruited from the Howard University Hospital (HUH) Division of Urology PCa patient population, the HUH PCa screening program, and the South Carolina PCa screening program. The PCa patients and controls were recruited between 2001 and 2005. Incident PCa cases in the current study were identified by an HUH urologist based on abnormal prostate-specific antigen (PSA) and/or digital rectal examination (DRE) as well as histological findings following a radical prostatectomy. Inclusion criteria of controls were men with PSA levels less than 4.0 ng/ml and/or normal DREs/biopsies. Tumor grade, ranging from 4-10, was collected for 62.0% of the cases (n=129). All study participants had available DNA extracted from whole blood and provided written informed consent for participation in genetic analysis studies under a protocol approved by the Howard University Institutional Review Board as well as from the HUH Division of Urology.

### Example 6

### CGEMS Project

This population consists of nationally available genetic data from 2,277 men of European-descent (488 non-aggressive cases, 688 aggressive cases and 1101 controls) collected through the NCI Prostate, Lung, Colon, and Ovarian (PLCO) Cancer Screening Trial. (Gohagan, 2000; Hayes, 2005), a randomized, well-designed, multi-center investigation sponsored and ran by the National Cancer Institute (NCI). Randomization for the PLCO Trial began in 1993 and ended in 2001 among men ages 55-74 years to evaluate the effect of screening on disease specific mortality, relative to standard care.

Men were included in the current analysis if they had a baseline PSA measurement before Oct. 1, 2003, completed a baseline questionnaire, returned at least one Annual Study Update (ASU), and had available SNP profile data through the Cancer Genetic Markers of Susceptibility (CGEMS) data portal (cgems.cancer.gov/). For PCa screening, blood samples were collected and men received a Prostate Specific Antigen (PSA) test and Digital Rectal Exam (DRE). Subsequent to the initial screen, participants received a PSA and DRE annually for three and five years, consecutively. Men who had PSA levels >4 ng/ml or abnormal DRE were referred to their health care provider for follow-up care.

Identification of PCa Cases and Controls.

The PLCO Trial identified 1176 incident PCa cases (488 non-aggressive and 688 aggressive) through various sources including: screening exams; reports from patients, physicians, or relatives; linkage with the National Death Index; or linkage with the state cancer registries. Incident PCa cases were pathologically confirmed with either aggressive (Gleason score ≧7 or tumor stage III/IV) or non-aggressive (Gleason score <7 or tumor stage I/II) disease based Gleason score and tumor stage at diagnosis. Since incident cases were defined as individuals diagnosed after the first year of follow-up, men receiving a diagnosis prior to one year of follow-up were excluded from the study.

Controls (n=1111) were matched to cases identified between 1993-2001 on age, time since initial screening, and year of blood draw using incidence density sampling. Incidence Density sampling accounts the dynamic nature of a cohort study. Under this selection strategy, controls were selected independently for each case from those who were at risk at the time of the diagnosis of the case. Identification as a control for a given case set was independent of the following: future diagnosis as a case, selection as a control for other case sets, and the number of entry and exit times. Therefore, individuals may be included as both a case and a control. The genotype data for individuals who were selected multiple times were taken into consideration for each selection. Other covariates that vary with time, such as age are defined differently each time, depending on the characteristics of the case set for which he was selected as a control.

Data Collection.

Access to clinical and background data collected through examinations and questionnaires was approved for use by PLCO. All participants signed informed consent documents approved by both the NCI and local institutional review boards. Access to clinical and background data collected through examinations and questionnaires was approved for use by the PLCO.

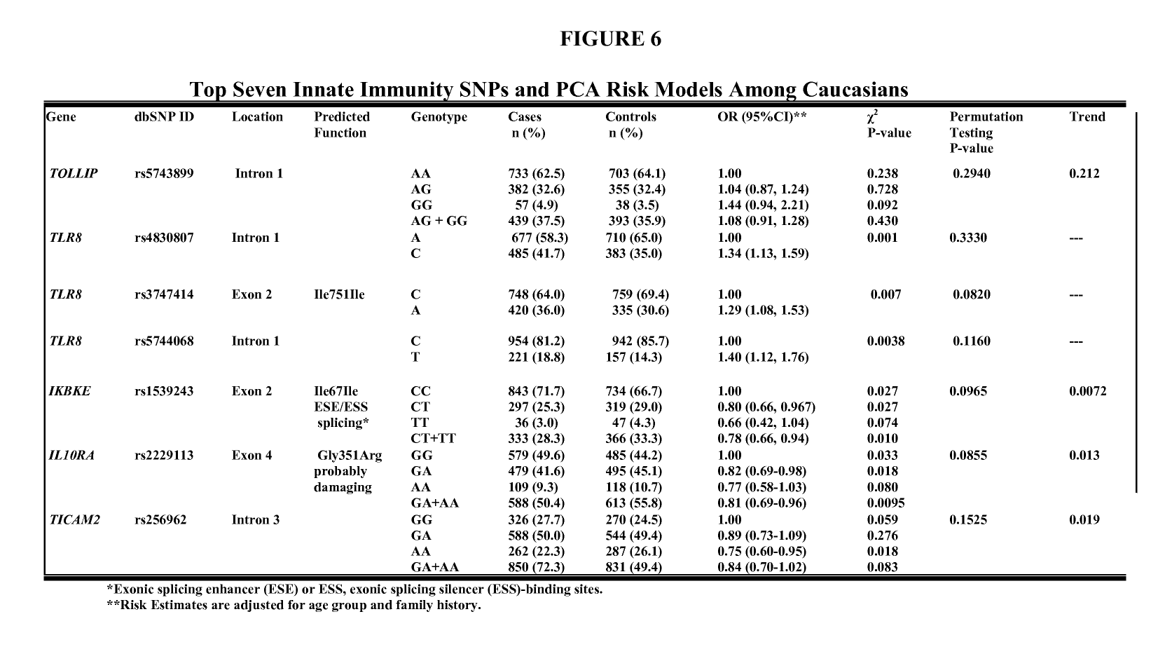

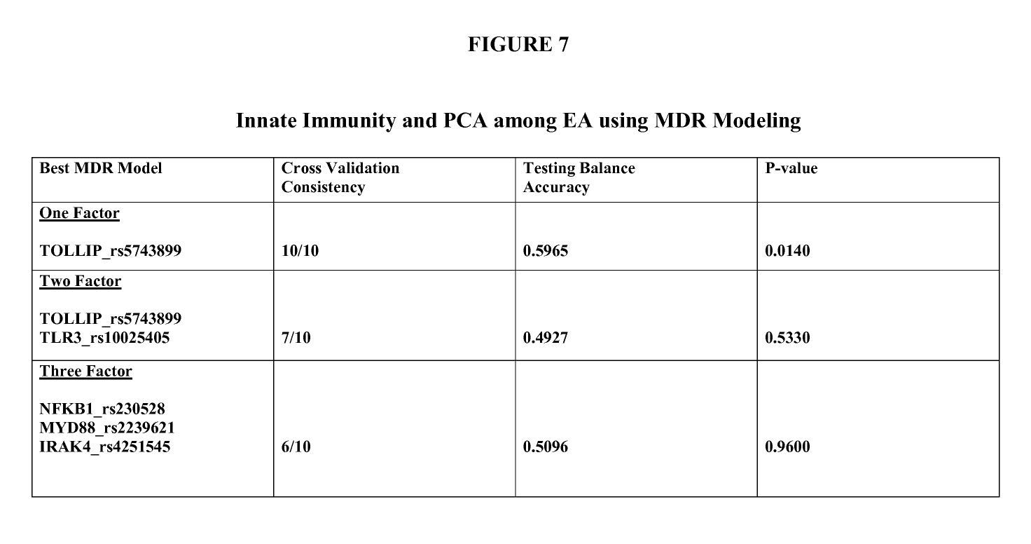

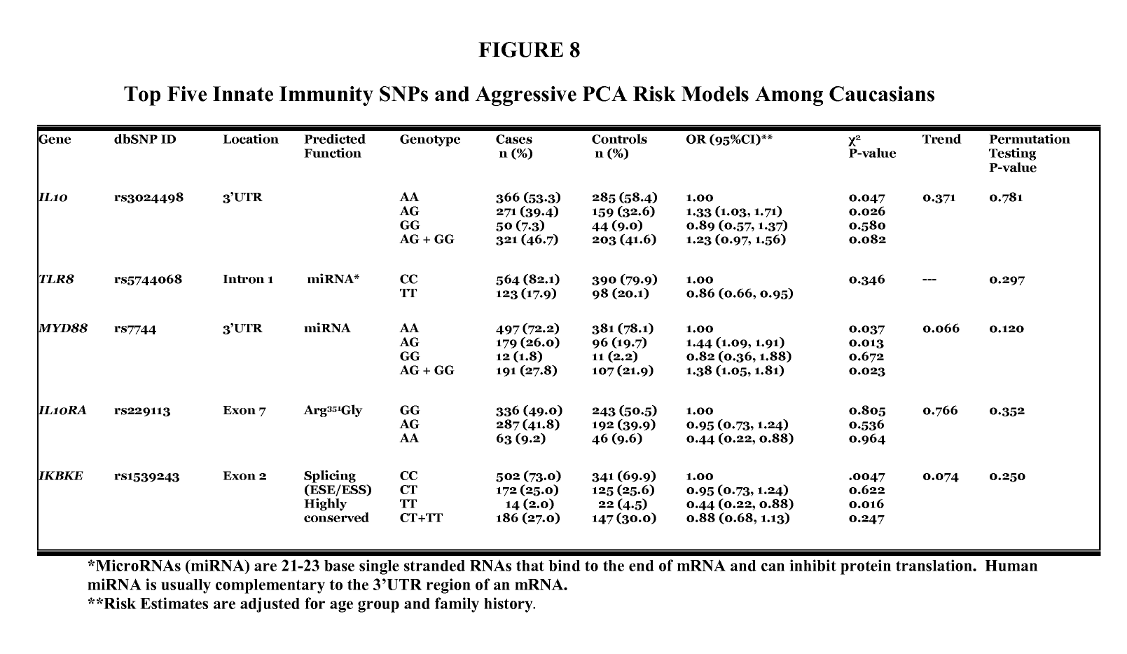

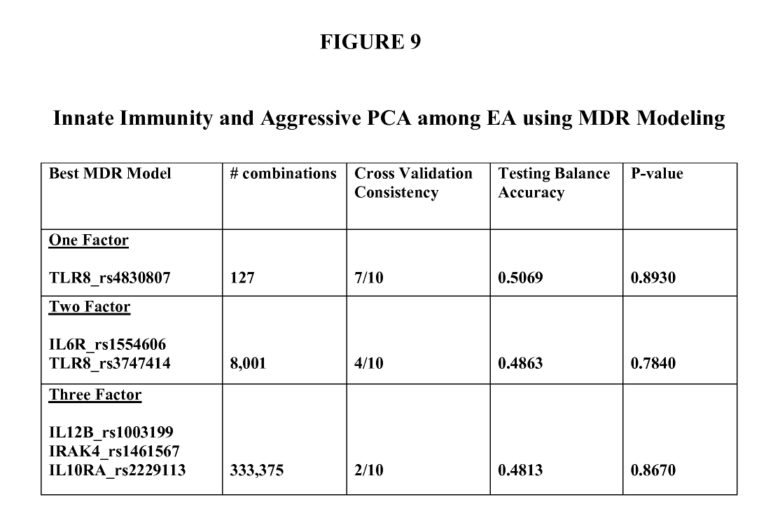

None of the innate immune response sequence variants were associated with prostate cancer outcomes among male participants of the CGEMS study after adjusting for multiple comparisons. In particular, TOLLIP rs5743899 SNP was not a significant predictor of prostate cancer risk or aggressive disease among men of European descent, as revealed in FIGS. 6-9. Unfortunately, TLR2 rs4696480 was not analyzed within the CGEMS data portal.

### Example 7

### PCa Consortia and Clinical Trial Ancillary Study (CTAS)

Patients diagnosed with PCa (1440 cases; 288 African-Americans and 1152 Europeans) are eligible for enrollment in the this study if they have histologically or cytologically confirmed adenocarcinoma of the prostate, are scheduled to undergo chemotherapy, are >18 years of age, have a life expectancy of greater than 12 weeks, and sign a written informed consent. Subjects will be excluded if they have uncontrolled intercurrent illness including, but not limited to, ongoing or active infection or psychiatric illness/social conditions that limits compliance with study requirements.

The study utilizes de-identified information and specimens collected from 200 Caucasian patients in the University of Louisville CTAS. The University of Louisville Clinical Trial Ancillary Study (CTAS) Database houses cancer patients' medical histories, demographic, and tumor characteristic data. For this study, patients undergo follow-up every 6-12 months for 5 years after the date of their last cancer treatment to secure information regarding the status of their disease. Available clinico-pathological data will include: (i) tumor-based properties (e.g., tumor pathology/grade/stage, size, number of lesions, nodal status, hormone receptor status, PSA); (ii) patient-related characteristics (e.g., age, self-identified race, first degree family history), cigarette smoking status (current, former, non-smoker, pipe/cigar only); cigarette smoke pack-years, number of cigarettes smoked per day; tumor behavior (e.g., tumor grade/stage/nodal/size/number); (iii) drug toxicity (e.g., liver dysfunction—(high serum transaminase, alkaline phosphatase, and/or bilirubin levels), kidney dysfunction (urinary drug metabolites); (iv) drug response (e.g., neutropenia, anemia, low platelet count, time-to-fluid retention, lesion size, lesion number, % PSA elevation); (v) tumor markers (e.g., hormone receptor status, PSA); and (vi) prognostic outcomes (e.g., PSA relapse, short time from treatment Nadir, time from Nadir to androgen independent PCa, PSA progression free survival, disease-free survival, progression-free survival, overall survival).

### Example 8

### Biospecimen Collection, Processing, Storage, and Analysis

Blood samples from CTAS are stored at −80° C. until further analysis. One set (two standard 3.2% sodium citrate-blue top-tubes) of samples of 5-10 ml of blood are drawn from CTAS participants by standard phlebotomy technique at study entry (i.e., prior to initiating a new therapy). Within 30 minutes after collection, blood samples are be stored on ice (at ˜0° C.). To ensure the integrity of macromolecules (i.e., protein and RNA) for ancillary studies (Wittliff et al., The Breast: Comprehensive Management of Benign and Malignant Disease, I. E. M. Copeland, Editor. 1998, W.B. Saunders Co: Philadelphia, Pa. p. 458-498), the samples are immediately centrifuged to isolate peripheral mononuclear blood cells (PMBCs) and stored at −80° C. until further processing. One sample is used for biomarker testing. The second sample will be separated and frozen and stored for future testing in case of problems with the first sample or its data. All patient identifiers will be removed and a unique subject number will be used for identification of samples.

DNA is extracted from blood samples (2.5 ml) using the AllPrep DNA/RNA/Protein Mini Kit (Qiagen) or the QIAamp DNA Mini Kit (Qiagen). DNA concentration is measured using the NanoDrop® ND-1000 Spectrophotometer. DNA samples are diluted to 75 ng/μl and stored at −80° C. until further analysis.

One hundred previously validated autosomal ancestry markers were included to account for potential population stratification among the admixed population of self-reported African-Americans, West African, East African, Afro-Caribbean, as previously described (Tian et al., Am. J. Hum. Genet., 2006. 79(4):640-649). Study participants were grouped from lowest to highest genetic West African Ancestry (WAA), with scores ranging from 0-100%. These 100 markers were assembled using DNA from self-identified African-Americans (Coriell Institute for Medical Research, n=96), Yoruban West Africans (HapMap, n=60), West Africans (Bantu and Nilo Saharan speakers, n=72), Europeans (New York City, n=24), and CEPH Europeans (HapMap Panel, n=60), as previously reported (Tian et al., Am. J. Hum. Genet., 2006. 79(4):640-649). All study participants are classified as men of African descent if they have a WAA score ranging between 25-100%. Men of European descent have a WAA score of less than 25%.

### Example 9

### Identification of Novel SNPs Among Micro-Array-Based Resequencing

Micro-Array Genome Selection: Germ-line DNA collected from 50 African-Americans (WAA ranging between 25-75%), 50 West Africans (WAA >75%), and 50 Caucasians (WAA <25%) are sequenced to detect novel and previously reported innate immunity-associated sequence using a new micro-array-based Genomic Selection (MGS) technology (Okou et al., Nat. Methods, 2007. 4(11):907-909). With MGS, oligonucleotide (oligo) arrays are used as a platform to “select” and isolate target DNA (tDNA) from specific regions of complex eukaryotic genomes. These regions, even if from different chromosomes, can be captured together and sequenced up to the limits of the oligo-based chip. In short, tDNAs are hybridized to long oligonucleotides (˜50 bp) then immobilized onto a high density microarray. Lastly, bound fragments are eluted off the microarray. The MGS process consists of the following steps: (i) fragmentation of human genomic DNA; (ii) hybridization of fragmented genomic DNA to a custom-designed high-density long oligonucleotide (50 bp) microarray; (iii) elution of tDNA fragments that bind to their complementary oligonucleotides on the microarray; (iv) amplification of eluted tDNA fragments using generic adapter primers previously ligated to tDNA fragments; and (v) hybridization of amplified fragments to a resequencing array to determine the genomic sequence. With this approach, it is possible to synthesize arrays with greater than 300,000 bp, each representing a different probe sequence.

Resequencing Array: Two 8 μm (each 300 kb) resequencing arrays (RAs) are processed using a standard Affymetrix GCS 3000 system. Unique genomic sequences from the latest human genome build of the innate immunity signaling pathway are obtained using the UCSC Table Browser function (Karolchik et al., Nucleic Acids Res, 2004. 32 (Database issue):D493-6) with repeats masked. These sequences are provided to chip design engineers at Affymetrix. Since genetic variants in regulatory elements far away from the coding sequence may influence the expression of a gene (Kleinj an and van Heyningen, Am J Hum Genet, 2005. 76(1):8-32), unique sequence upstream and downstream of the target genes are also included. Resequencing strategies are based on aligning the DNA sequence of a given specie genome to a reference sequence of the same species, to identify the bases that are the same and those that are different between the two sequences. In chip-based resequencing, the reference sequence is immobilized on a chip.

Chip Hybridization, Scanning and Basecalling: The hybridization and scanning process consists of three steps: (1) hybridization, (2) chip wash/scanning, and (3) basecalling with RATools. Prior to the process, samples will be quantified and pooled, fragmented with DNAse, and labeled with biotin. Samples will be hybridized to RAs, washed and scanned using established protocols (Cutler et al., Genome Res, 2001. 11(11):1913-25; Zwick et al., Genome Biol, 2005. 6(1):R10). After the chips are scanned, RATools will use the raw image file to perform basecalling. To obtain very high quality (≧Phred 50) data, the thresholds are set high and it is predicted that 90% of the 300 kb bases on the RA, or 270 kb per individual will be called. A total of about 25 chips are used for the project. DNA sequences obtained are used to inform and improve the tDNA production protocols further. For example, potential ligation biases in the MGS protocol are identified enabling improvements to the assay.

Estimate of Genetic Variation: The total number of SNPs expected in the controls can be estimated as the product of Watterson's estimate of theta (5.32E-4) (Watterson et al., Am J Drug Alcohol Abuse, 1975. 2(1):99-111), the natural log of the sample size [ln(50)], and the length of sequence screened (270,000). Performing this calculation predicts that 562 SNPs per 300 kb of innate immunity genes will be identified among controls. Since all 25 innate immunity genes are resequenced, approximately 1,124 SNPs are expected to be detected. An identical SNP number in cases under the null model of no differences between cases and controls is expected. The neutral theory predicts that 54% of SNPs are rare, with allele frequencies <1% considered rare, which will not be considered in the current proposal. The remaining 46% SNPs with allele frequencies >1%, are considered common SNPs. Most of the 517 common SNPs are expected to be found in both cases and controls.

### Example 10

### Selection of Resquencing SNPs

Innate immunity-related SNPs are selected from the resequencing analysis based on one or more of the following criteria: (i) minor allele frequency >1%; (ii) non-synonymous SNPs, since they may alter protein expression, function, or structure; (iii) marked disparities in genotype frequency comparing men of African or Asian descent to their Caucasian counterparts; (iv) published reports demonstrating a statistically significant relationship between selected SNPs and inflammatory related diseases; (v) commonly studied loci in the literature; (vi) evidence demonstrating a link with alterations in mRNA expression/stability or protein expression, structure, or function. The LD between SNPs is also calculated in this dataset to assess the coverage of the selected genes with the goal of capturing most of the common variations as previously detailed (Bailey et al., J Dev Behav Pediatr, 2000. 21(5):315-21). Genotyping of the finalized candidate SNPs list will involve high-throughput genotyping strategies detailed below.

### Example 11

### SNP Profiling for Using TaqMan PCR, SNPstream, and Illumina Veracode System

In order to evaluate and validate innate immunity markers recovered from microarray-based resequencing as predictors of PCa outcomes, disease progression and prognosis, de-identified germ-line DNA from PCa cases and disease-free individuals undergo high-throughput SNP analysis using a 48-plex SNPStream, TaqMan PCR, or Illumina's Veracode System. Since SNPStream has an 80% success rate on array designs, TaqMan PCR is used to recover targets not analyzed using the later strategy. Allelic discrimination will focus on a combination of approximately 240 innate immunity related genes among MADCaP, HUPCS, and CTAS samples.

### Example 12

### Screening for Single Gene Markers Predictive of PCa Risk Using the Chi-Square Test and Logistic Regression Analysis

Logistic regression analysis was used to evaluate 127 and 18 innate immune response associated SNPs among men of European and African descent, respectively, in relation to prostate cancer outcomes. To assess whether individuals possessing at least one minor apoptotic allele influence the risk of developing PCa, we tested for significant differences in the distribution of homozygous wildtype, heterozygous, or homozygous variant high genotypes/haplotypes between cases and controls using the chi-square test of heterogeneity. A case-case analysis was used to evaluate the relationship between apoptosis-related alleles and aggressive PCa among men of European. In this exploratory investigation, we determined the distribution and inheritance of apoptosis-related genes comparing men with high tumor grade (Gleason score ≧7) to those with a lower grade of disease (Gleason score <7). The associations between PCa outcomes and selected polymorphic genes, expressed as odds ratios (ORs) and corresponding 95% confidence intervals (CIs), will be estimated using unconditional multivariate LR models adjusted for potential confounders. Risk estimates for men of European descent were adjusted for age-group and family history of prostate cancer for men; whereas models for men of African descent were adjusted for age (years) and West African ancestry. LR analysis for genetic variants and PCa development were conducted using the wild-type or common genotype as referent category. All chi-square test and LR analyses will be conducted using SAS 9.2 (SAS Institute Inc, Cary, N.C.). Statistical significance will be assessed using a P-value <0.05. Adjustments for multiple comparisons were made using bonferroni correction and permutation testing.

### Example 13

### Evaluation of Individual and Joint Modifying Effects using MDR Analysis

MDR 1.0.0 (SourceForge, Inc., sourceforge.net) will be used to further evaluate main effects as well as gene-gene interactions associated with PCa risk. MDR has been described and reviewed. Briefly, it is a “model free” method (it does not assume a specified genetic model) and “nonparametric” (it does not estimate parameters) for detecting and characterizing high-order interactions in observational studies. With MDR, multi-locus genotypes will be pooled into high-risk and low-risk groups, reducing high-dimensional data to a single variable dimension and permitting an investigation of gene-gene and gene-environment interactions. Concisely, this one-dimensional multi-locus genotype variable will be evaluated for its ability to classify and predict PCa or aggressive disease through cross-validation and permutation testing. Among all of the gene-gene combinations, a single model will be selected that maximizes the case-to-control ratio of the high-risk groups while minimizing classification and prediction errors. To evaluate how many times the same MDR model will be identified in each possible 9/10^{ths} of the data, the average cross-validation consistency from the observed data will be compared to the distribution of the average consistencies under the null hypothesis of no association to be derived empirically from 1,000 permutations. This approach accounts for multiple testing issues as long as the entire model-fitting procedure is repeated for each randomized dataset to provide an opportunity to identify false-positives. The MDR software is open-source and freely available online. LR modeling was used to calculate risk estimates of risk of developing PCa affiliated with interaction models identified by MDR. The power of MDR for identifying gene-gene interactions in the presence of common sources of noise has been previously evaluated, and shown to be excellent in the majority of cases even for small sample sizes (200 cases and 200 controls).

### Example 14

### Estimated Statistical Power Associated with Single SNP and Haplotype Effects Using Parametric Statistical Modeling

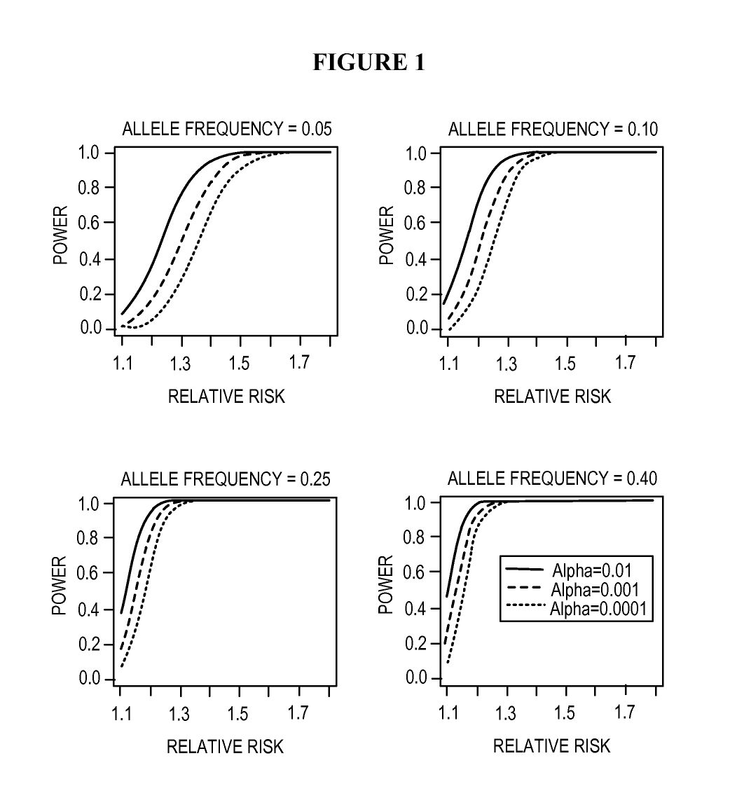

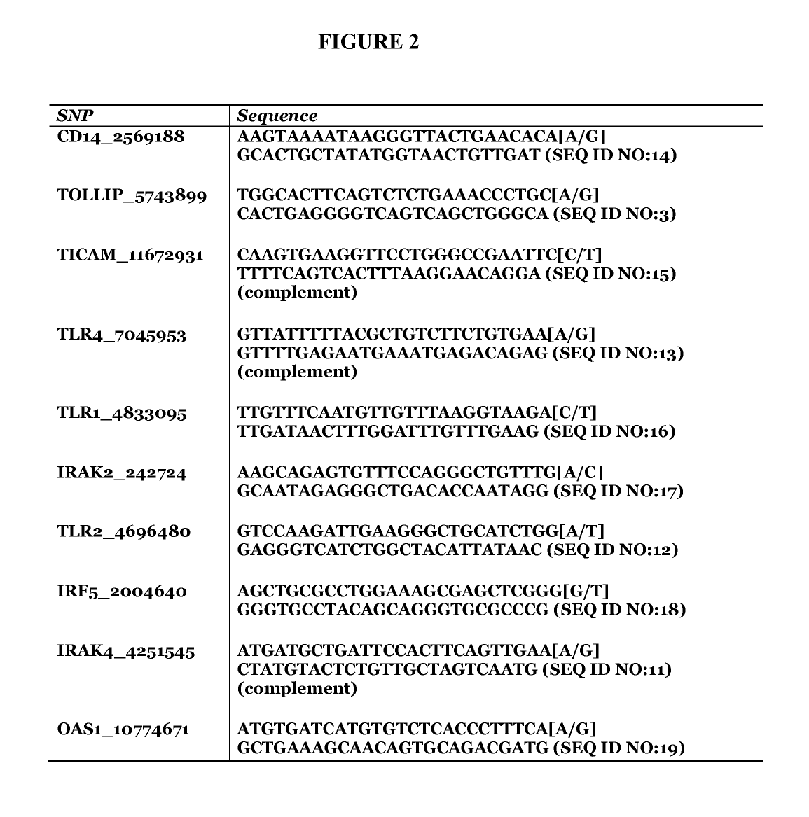

This study examines single loci and haplotype effects in relation to prostate cancer susceptibility among study participants using parametric statistical modeling, including logistic regression modeling. The expected power of this study can be estimated by specifying values for a number of parameters (e.g., minor allele frequency, disease prevalence, genotype relative risk) in order to perform the relevant calculations. Calculations to determine the power of detecting significant relationships between variant innate immunity-related SNPs and PCa outcomes were conducted, assuming the outcome was in complete linkage disequilibrium with an innate immunity-predisposing variant. For the prostate cancer case-control study, the sample is made of 2880 cases and 2880 controls. The power of the sample to observe relative risk is estimated between 1.1-1.8 and a minor allele frequency between 0.05-0.40 using a significance level of 0.05. To provide coarse adjustments for multiple testing, the power for each model was determined assuming both a liberal adjusted type-I error rate of α=0.01, as well as more conservative adjusted type-I error rates of α=0.001 and 0.0001. The power tables for the sample under different genetic models and different type I error rates are shown in FIG. 1. As expected, power increases with an increase in both the SNP minor allele frequency and the relative risk values. Assuming a relative risk of >1.2 (which is reasonable for a disease-influencing variant) and a reasonable type-I error rate of 0.001, >80% power to detect significant outcomes relative to variant innate immunity markers is estimated, if the minor allele has a frequency ranging from ≧25% to 40%. Power of the sample should be further augmented by the use of haplotypes rather than single SNPs for logistic regression modeling. Because of these results, the proposed sample has sufficient size to detect risk estimates under a variety of plausible genetic models, even after correcting for multiple testing.

### Example 15

### Innate Immunity-Related Sequence Variants as Predictors of Breast Cancer Risk Among Women of African Descent

The individual effects of 14 innate immunity genes (TLR 1-4, CD14, IRAK2, IRAK4, IRF3, IRFS, MYD88, OAS1, TICAM1, TOLLIP, and RNAseL) in relation to breast cancer and tumor behavior among 200 African-American female patients was evaluated. Twenty genetic alterations were evaluated in germ-line DNA samples collected from 108 patients and 92 disease-free individuals using SNPstream. All study participants were recruited from Grady Memorial Hospital and Emory Midtown Hospital in Atlanta, Ga. Independent effects of 20 variant innate immunity genes were studied in relation to BCa risk and tumor characteristics (tumor stage/size/pathology, hormone receptor status, and HER-2 neu status) using case-control and case-only study designs, respectively. Inheritance of the IRAK4 rs4251525 TT genotype was associated with a statistically significant two-fold increase in BCa risk [OR=2.23; 95% CI=1.24, 3.98; p=0.007; p-trend=0.0021] (Table 3). None of the markers were associated with advanced disease or other tumor characteristics. A non-synonymous sequence variant detected in IRAK4 (Ala→Thr) may serve as an important BCa detection tool among women of African descent. Individuals inheriting high-risk innate immunity loci (linked with elevated inflammatory response or cell survival) may have an increased risk of developing BCa or aggressive tumor subtypes relative to those with the referent genotypes.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

TABLE 3
Association between Innate Immunity Sequence Variants and Breast Cancer Risk

| SNP_rs number | Sequence | OR (95% CI) | P-value | P-trend | |
|---|---|---|---|---|---|
| TLR4_7045953 | GTTATTTTTACGCTGTCTTCTGTGAA[A/G] | | | | marginal |
| TT | GTTTTGAGAATGAAATGAGACAGAG | 1.00 | 0.0698 | 0.043 | |
| CT | (SEQ ID NO: 13) (complement) | 1.97 (1.08, 3.58) | | | |
| CC | | 1.8 (0.60, 5.44) | | | |
| CT + CC | | 1.95 (1.10, 3.44) | 0.0213 | | |
| TLR2_4696480 | GTCCAAGATTGAAGGGCTGCATCTGG[A/T] | | | | marginal |
| TT | GAGGGTCATCTGGCTACATTATAAC | 1.00 | 0.040 | 0.206 | |
| TA | (SEQ ID NO: 12) | 0.47 (0.25, 0.87) | | | |
| AA | | 0.81 (0.36, 1.84) | | | |
| TA + AA | | 0.53 (0.30, 0.94) | 0.030 | | |
| IRAK4_4251545 | ATGATGCTGATTCCACTTCAGTTGAA[A/G] | | | | |
| Asn > Ser | CTATGTACTCTGTTGCTAGTCAATG | 1.00 | 0.006 | 0.0021 | |
| CC | (SEQ ID NO: 11) (complement) | 1.89 (1.04, 3.46) | | | |
| CT | | 7.13 (1.53, 33.3) | | | |
| TT | | 2.23 (1.24, 3.98) | 0.007 | | |
| CT + TT | | | | | |
| IRAK2_6442161 | GAGATAAGGCAGGAGGCCATTTACAG[C/T] | | | | marginal |
| CC | AGTCTTGGGGAGGCTGAATCAGAGC | 1.00 | 0.046 | 0.016 | |
| CT | (SEQ ID NO: 10) | 0.63 (0.35, 1.14) | | | |
| TT | | 0.29 (0.10, 0.83) | | | |
| CT + TT | | 0.56 (0.32, 0.99) | 0.044 | | |
| IRF3_7251 | CATGGATTTCCAGGGCCCTGGGGAGA[C/G] | | | | marginal |
| CC | CTGAGCCCTCGCTCCTCATGGTGTG | 1.00 | 0.051 | 0.019 | |
| CG | (SEQ ID NO: 9) | 0.65 (0.36, 1.19) | | | |
| GG | | 0.30 (0.11, 0.84) | | | |
| CG + GG | | 0.58 (0.32, 1.03) | 0.061 | | |
| TLR3_10025405 | TCACAGACTCAGGAGATGGCGTTGGC[A/G] | | | | |
| AA | AAATCACTTGGTCCCACTGGGATTC | 1.00 | 0.018 | 0.025 | |
| AG | (SEQ ID NO: 8) | 0.84 (0.47, 1.53) | | | |
| GG | | 0.20 (0.06, 0.65) | | | |
| AG + GG | | 0.69 (0.39, 1.21) | 0.190 | | |

What is claimed is:

1. A method of determining a subject's risk of developing prostate cancer (PCa), the method comprising detecting the presence or identity of a haplotype in a sample from the subject, wherein the haplotype comprises one or more of:
an “A” allele at rs4696480; a “G” allele at rs5743899; a “C” allele at rs4830807; a “T” allele at rs230528; or a “C” allele at rs5743808,
wherein the presence or identity of the haplotype indicates that the subject has an increased risk of developing PCa.

2. The method of claim 1, wherein detecting the presence or identity of a haplotype comprises:
obtaining a sample comprising DNA from the subject; and
determining the identity, presence, or absence of the alleles in the sample.

3. The method of claim 2, wherein the sample is obtained from the subject by a health care provider.

4. The method of claim 2, wherein the sample is provided by the subject without the assistance of a health care provider.

5. The method of claim 1, wherein the subject is of African descent.

6. The method of claim 1, wherein the subject has a West African Ancestry score of 25% or greater.

7. The method of claim 1, wherein the haplotype is an “A” allele at rs4696480.

8. The method of claim 1, wherein the presence of a “G” allele at rs5743899.

9. A method of determining a subject's risk of developing breast cancer (BCa), the method comprising detecting the presence or identity of a haplotype in a sample from the subject, wherein the haplotype comprises one or more of:
an “A” allele at rs10025405; a “T” allele at rs4696480; a “T” allele at rs4251524; a “C” allele at rs7045953; a “C” allele at rs6442161; or a “C” allele at rs7251,
wherein the presence or identity of the haplotype indicates that the subject has an increased risk of developing BCa.

10. The method of claim 9, wherein detecting the presence or identity of a haplotype comprises:
obtaining a sample comprising DNA from the subject; and
determining the identity, presence, or absence of the alleles in the sample.

11. The method of claim 10, wherein the sample is obtained from the subject by a health care provider.

12. The method of claim 10, wherein the sample is provided by the subject without the assistance of a health care provider.

13. The method of claim 9, wherein the subject is of African descent.

14. The method of claim 9, wherein the subject has a West African Ancestry score of 25% or greater.
